(19) United States
(12) Patent Application Publication
(10) Pub. No.: US 2011/0212467 A1
(43) Pub. Date: Sep. 1, 2011

(54) INHIBITORS OF dUTPase

(75) Inventors: Robert D. Ladner, Santa Monica, CA (US); Nouri Neamati, Fullerton, CA (US)

(73) Assignee: UNIVERSITY OF SOUTHERN CALIFORNIA, Los Angeles, CA (US)

(21) Appl. No.: 13/061,068

(22) PCT Filed: Aug. 27, 2009
(86) PCT No.: PCT/US09/55264

§ 371 Date: May 13, 2011

(30) Foreign Application Priority Data
Aug. 27, 2008 (US) 61/092330

(51) Int. Cl. G01N 33/573; C12N 5/09; C07D 261/16; C07D 417/12; C07D 285/125; C07D 473/04

(52) U.S. Cl. 435/7.8; 435/375; 548/245; 548/171; 548/136; 544/266

(57) ABSTRACT

Evidence demonstrating that elevated expression of dUTPase protects breast cancer cells from the expansion of the intracellular uracil pool, translating to reduced growth inhibition following treatment with 5-FU is provided. The implementation of in silica drug development techniques to identify and develop small molecule inhibitors of dUTPase are reported. As 5-FU and the oral 5-FU pro-drug capecitabine remain central agents in the treatment of a variety of malignancies, the clinical utility of a small molecule inhibitor to dUTPase represents a viable strategy to improve the clinical efficacy of these mainstay chemotherapeutic agents.

### STATEMENT REGARDING FEDERALLY SPONSORED RESEARCH OR DEVELOPMENT

This invention was made with government support under Contract No. R21 5R21CA104796-3 awarded by the National Institutes of Health. The government has certain rights in the invention.

### FIELD OF THE INVENTION

The present invention relates to the utilization of small molecule inhibitors of deoxyuridine triphosphosphate nucleotidohydrolase (dUTPase) for the treatment of various diseases including but not limited to human cancers, immune disorders including rheumatoid arthritis and inflammatory bowel disease as well as viral, parasitic and bacterial infection.

### BACKGROUND OF THE INVENTION

### The Role of Uracil-DNA Metabolism in Cancer Chemotherapy

Thymidylate metabolism has long been an important target for widely utilized chemotherapeutic agents (i.e. the antifolates and fluoropyrimidines) that provide benefit in the treatment of leukemias, head and neck, breast and gastrointestinal cancers (51). The major mechanism of action of this class of antineoplastic drugs is the inhibition of enzymes that mediate critical steps in thymidylate metabolism. The de novo biosynthesis of thymidine monophosphate (TMP) occurs by the reductive methylation of dUMP by the enzyme thymidylate synthase (TS) to yield TMP, which is then converted to thymidine triphosphate (TTP) for DNA replication as shown in FIG. 1. The methyl donor in this reaction, 5, 10 methylenetetrahydrofolate (MTHF) is oxidized to dihydrofolate (DHF), and the levels of MTHF are maintained during TMP synthesis by the combined actions of dihydrofolate reductase (DHFR) and serine hydroxymethyltransferase (SHMT), FIG. 1. The pool of dUMP required for the TS reaction is supplied by two sources; deamination of dCMP by deoxycytidylate deaminase (DCD) and reduction of UDP by ribonucleoside diphosphate reductase (RNR). dUDP generated by the RNR reaction is phosphorylated to dUTP by nucleoside diphosphate kinase (NDP kinase) and is rapidly hydrolyzed to dUMP by deoxyuridine triphosphate nucleotidohydrolase (dUTPase). Thymidine kinase (TK) mediates the primary salvage pathway for thymidine nucleotides.

Inhibitors of the TS reaction include members of the fluoropyrimidine class of anticancer agents, such as 5-fluorouracil (5-FU), fluorodeoxyuridine (FUdR) and novel 5-FU pro-drugs such as capecitabine (Xeloda). These agents inhibit TMP biosynthesis by forming a ternary complex between the active metabolite (FdUMP), the methyl donor (MTHF) and the TS enzyme (FIG. 2.) (51). Novel folate-based TS inhibitors such as ZD1694 (Tomudex, Raltitrexed) and ZD9331 have also been developed and have proven to be highly specific and potent inhibitors of TS (53). The TS reaction can also be inhibited indirectly by targeting DHFR with antifolate agents such as methotrexate and metoprine. Inhibition of the DHFR enzyme blocks TMP production by limiting the availability of the MTHF cofactor required for the TS reaction as illustrated in FIG. 2.

Although broadly utilized, the clinical utility of TS and DHFR as therapeutic targets is hindered by the high incidence of resistance (either intrinsic or acquired) observed in many cancers. Since drug resistance is a common phenomenon, understanding mechanisms of action and predicting response and overall patient outcome is of considerable importance (59). Studies attempting to elucidate the molecular mechanisms of cell killing mediated by inhibitors of TS and DHFR suggest that cytotoxicity results from a process termed “thymineless death”. Cell death initiated by thymineless conditions was originally postulated to result from DNA synthesis arrest subsequent to extreme TTP pool depletion (47). However, more recent investigations suggest that multiple factors contribute to the underlying mechanism of thymineless death, particularly imbalance of other dNTP pools. The detrimental consequences of nucleotide pool imbalance and thymineless conditions have since been reviewed (42, 43, 47, 54, 55). One consequence of TS inhibition by anti-cancer agents that has gained acceptance involves aberrant uracil-DNA metabolism. There is a growing body of evidence suggesting that dUTP accumulation, uracil misincorporation into DNA, and uracil-DNA glycosylase-induced strand breaks, are important mediators of cytotoxicity resulting from inhibition of thymidylate metabolism. The biochemical basis of uracil-DNA-mediated cell death lies in the fact that uracil is not a native component of DNA in virtually all known organisms. Uracil can arise in DNA either by the spontaneous deamination of cytosine residues or through dUTP utilization by DNA polymerases during replication and repair (45, 57, 58). Since cytosine deamination can lead to G:C to A:T transition mutations, the cell has evolved highly efficient mechanisms to facilitate the exclusion of uracil from DNA (57). When uracil does occur in DNA, uracil-DNA glycosylase (UDG) initiates the base-excision repair pathway to remove and correct the misincorporated nucleotide. In order to prevent dUTP utilization during DNA replication, the enzyme dUTPase hydrolyzes dUTP to yield dUMP and pyrophosphate. This reaction effectively eliminates dUTP from the DNA biosynthetic pathway and also provides substrate (dUMP) for the de novo synthesis of thymidylate. Under normal cellular conditions, the maintenance of uracil-free DNA is achieved through the combined actions of dUTPase and UDG.

Although dUTP is a normal intermediate in thymidylate biosynthesis, its extensive accumulation and misincorporation into DNA is lethal in both prokaryotic and eukaryotic organisms (26, 48). The exact mechanism for uracil-DNA-mediated cell death has not been definitively proven, however there is compelling evidence suggesting that UDG-initiated repair is a central component of this process. For example, inactivation of dUTPase in E. coli results in the dramatic accumulation of dUTP pools leading to extensive uracil misincorporation during replication. Under conditions of elevated dUTP pools, the cell engages in repeated cycles of uracil misincorporation and UDG-mediated repair. This iterative process results in increased recombination, DNA strand breaks, and ultimately cell death as illustrated in FIG. 2. (26, 48, 56, 60).

A similar phenomenon involving aberrant uracil-DNA metabolism is thought to occur during inhibition of de novo thymidylate metabolism by anti-cancer agents (44, 49, 50, 52, 56, 63, 64, 65, 66). Inhibition of the TS reaction, in certain cell lines, results in the depletion of TTP pools while cellular dUMP pools accumulate behind the metabolic blockade due in part to a loss of feedback control on DHFR, deoxycytidine deaminase (DCD) and thymidine kinase (TK) (64). Subsequently, dUTP pools accumulate due to the action of mono- and di-phosphate kinases. Once levels of dUTP accumulate beyond a threshold level, over-saturating cellular dUTPase activity, the dUTP/TTP ratio increases. Because DNA polymerases do not distinguish between uracil and thymine bases, dUTP is misincorporated into replicating DNA resulting in uracil-DNA-mediated cytotoxicity as illustrated in FIG. 2. Considering the critical roles of dUTPase, UDG and DCD in the uracil-misincorporation pathway, variation in the expression of these enzymes within target cells may have substantial implications toward the efficacy of TS-directed chemotherapy.

Cancer Treatment using 5-fluorouracil (5-FU)

The fluoropyrimidine 5-FU is widely used in the treatment of a range of cancers, including breast cancers, and cancers of the aerodigestive and gastrointestinal tract (1). However, 5-FU has had the greatest impact and is arguably the most successful drug approved to date for the treatment of colorectal cancer (CRC). Throughout 50 years of clinical development, the response rate of advanced CRC chemotherapy using 5-FU and 5-FU-based combinations has improved from 10-15% to 40-50%, primarily due to the introduction of efficacious combination partners such as the topoisomerase I inhibitor irinotecan and the platinum agent oxaliplatin and determination of optimal drug scheduling and administration (2-4). Biological agents, such as the monoclonal antibodies cetuximab which targets the epidermal growth factor receptor (EGFR) and bevacizumab an inhibitor of vascular endothelial growth factor (VEGF), have recently demonstrated additional clinical benefit when included in 5-FU-based regimens in metastatic CRC (mCRC) through suppression of receptor-mediated tumor processes (5, 6). However, despite these improvements, approximately one-half of patients treated with 5-FU-based therapies will derive no benefit, highlighting the need for the identification of therapeutic targets and strategies to overcome the frequent occurrence of drug resistance and improve current 5-FU-based therapies.

5-FU continues to remain the mainstay of therapeutic regimens employed in the treatment of CRC and other gastrointestinal malignancies. However, 5-FU continues to prove itself an efficacious agent in additional cancers including breast cancer. Two pivotal clinical trials have recently reported capecitabine to be an efficacious combination partner for targeted agents approved for breast cancer (7, 8).

The Role of dUTPase as a Modulator of Chemosensitivity to 5-FU-Based Therapies

dUTPase is a ubiquitous enzyme that is essential for viability in both prokaryotic and eukaryotic organisms (26, 48). As the main regulator of dUTP pools, the expression of dUTPase could have profound effects on the utility of chemotherapeutics that inhibit thymidylate biosynthesis. Normally, dUTPase mediates a protective role by limiting the expansion of dUTP pools and countering the cytotoxic effect of uracil misincorporation. According to this model, elevated levels of dUTPase could prevent TS inhibitor-induced dUTP accumulation and induce drug resistance. Convincing evidence supporting dUTPase enzyme activity as an important determinant of drug-induced cytotoxicity was first provided by a series of articles published by Canman, et al. (33, 46). Resistance to FUdR in SW620 colon cancer cells (SW620) correlated with elevated levels of dUTPase in comparison to HT29 cells. SW620 cells possessed 4.4-fold more dUTPase activity than the HT29 cells, and did not accumulate dUTP pools during exposure to FUdR. The authors speculated on the importance of dUTPase expression in modulating sensitivity, however, comparison of non-isogenic cell lines left doubt as to the actual mechanism of toxicity in each cell line. In later work, this group ectopically over-expressed the E. coli dUTPase in FUdR-sensitive HT29 cells and measured the response to the TS inhibitor FUdR. The manipulated cell lines (dutE1 and dutE7) contained dUTPase activity 4 to 5-fold higher than the neo-transfected controls (con2 and con3). It was concluded that the overexpression of dUTPase conferred protection from FUdR-induced DNA strand breaks and increased viability over control cells at 24 hours post-drug exposure. In subsequent analysis, the isogenic cell lines generated by Canman were tested for their sensitivity to the TS inhibitor CB3717 and the DHFR inhibitor methotrexate (MTX). Similar results were obtained, where dUTPase over expression resulted in a significant decrease in dUTP accumulation and increased resistance to drug treatment when compared to the controls (28, 62). Utilizing RNAi technology, we have extended these observations and demonstrate that lowered expression of dUTPase sensitizes human cancer cell lines to the cytotoxic effect of TS inhibition.

Rationale and Therapeutic Potential of Uracil-DNA Metabolism in Cancer Therapeutics

Chemotherapeutic agents that target de novo thymidylate metabolism are critical for the treatment of a variety of solid tumors, however clinical efficacy is often hindered by drug resistance. Because resistance to these agents is a common occurrence, the identification and exploitation of novel determinants of drug sensitivity within this pathway of proven therapeutic utility is extremely important. The inventors have successfully demonstrated that uracil-DNA misincorporation pathway can play a driving role in mediating cytotoxicity to TS-directed chemotherapies. Considering the central role of dUTPase, the inventors believe that directly inhibiting dUTPase with novel agents will promote dUTP accumulation and the uracil-misincorporation pathway thereby improving the clinical efficacy of TS-directed chemotherapy.

The major significance of the research lies in the fact that the treatment of colon, breast and lung cancers, utilize thymidylate synthase (TS) inhibitors as keystone members of combination therapy. The TS inhibitor 5-FU remains the foundation of all first and second line regimens in the treatment of colon cancer. No other single agent, including oxaliplatin, irinotecan, Erbitux and Avastin, demonstrates as much activity in colon cancer as 5-FU. In addition to colon cancer, TS-directed agents have demonstrated efficacy in several other solid tumor types. The results of a recent clinical trial reported at ASCO (note: please indicate what ASCO means) strongly support the utility of TS-directed therapies for the treatment of breast cancer. Dr. Charles E. Geyer from Allegheny General Hospital reported impressive results with the combination of capecitabine (Xeloda—an oral pro-drug of 5-FU), and lapatinib (a dual specificity inhibitor of EGFR and Her2). This combination demonstrated a 12.2% improved response rate compared to capecitabine alone in patients with advanced breast cancer who where ErbB2 positive and previously failed multiple lines of therapy. In addition to this, time to progression increase from 19.7 to 36.9 weeks with the combination therapy (P=0.00045). TS-directed therapies are also utilized in the treatment of lung cancer, where Pemetrexed (Alimta) represents an important component of combination chemotherapy for non-small cell lung cancer (NSCLC). Thus, the significance of TS-directed chemotherapy in cancer therapeutics is profound and efforts to improve the efficacy of TS inhibitors have broad and significant implications.

One goal of these studies was to understand the role of the uracil-DNA misincorporation pathway in mediating cytotoxicity during TS inhibition and to generate a strong, mechanism-based rationale to target this pathway through drug development in order to significantly improve response to TS-directed chemotherapy in the clinic. Evidence generated by our laboratory in yeast and cell line-based models demonstrate that dUTP accumulation and uracil misincorporation into DNA can be a critical determinant of toxicity initiated by TS-directed therapy. dUTPase is the main regulator of dUTP pools and elevated expression of this enzyme abrogates the uracil misincorporation pathway, leading to resistance to TS-directed chemotherapy. Our data demonstrate that SW620 colon cancer cells are 70-fold more sensitive to TS inhibitors when dUTPase levels are lowered by siRNA resulting in dUTP accumulation and uracil, misincorporation, and enhanced DNA damage and cytotoxicity (9). Furthermore, elevated dUTPase expression in colon cancer is associated with non-response to 5-FU-based chemotherapy (12). The clinical significance of this is based on the belief that inhibition of dUTPase and exploitation of the uracil-DNA misincorporation pathway can significantly increase the efficacy of TS-directed agents leading to improved response rates in multiple common cancers. Current pre-clinical in vitro data validating this pathway are promising and suggest that the cellular capacity to accumulate dUTP pools is a key determinant of chemosensitivity (9, 32).

Rationale for dUTPase as a Target for Drug Development

Significant effort has been directed towards defining mechanisms of cytotoxicity and resistance to agents that inhibit thymidylate metabolism such as 5-fluorouracil (5-FU). Evidence from these investigations suggests that the accumulation of excessive dUTP pools and iterative misincorporation of dUMP into DNA may be key mediators of cytotoxicity subsequent to inhibition of de novo thymidylate biosynthesis. As the sole regulator of dUTP pools, dUTPase represents a novel intervention point that alone or in combination with TS inhibitors could improve the clinical efficacy of this important class of chemotherapeutic agents. In addition to the pathway driven rationale for dUTPase as a therapeutic target, several other practical advantages make this enzyme particularly amenable to drug development:
1) dUTPase has a well characterized, non-redundant cellular function.
2) Crystallographic data of enzyme-substrate complexes are available at high resolution.
3) The biochemical consequences of dUTPase inhibition have measurable outcomes that can be used to validate the mechanism of drug action (61).

### SUMMARY OF THE INVENTION

Deoxyuridine triphosphate nucleotidohydrolase (dUTPase) catalyzes the hydrolysis of deoxyuridine triphosphate (dUTP) to deoxyuridine monophosphate (dUMP) and pyrophosphate (PPi) providing substrate for thymidylate synthase (TS) and DNA synthesis and repair. Although dUTP is a normal intermediate in DNA synthesis, its accumulation and misincorporation into DNA as uracil is lethal. Importantly, uracil misincorporation represents an important mechanism of cytotoxicity induced by the TS-targeted class of chemotherapeutic agents including 5-FU. A growing body of evidence suggests that dUTPase is an important mediator of response to TS-targeted agents.

The present invention demonstrates that induced dUTPase expression in a tet-repressible MCF-7 breast cancer cell line suppresses dUTP pool expansion and increases resistance to 5-FU. Importantly, the present invention also indicates that dUTPase expression in breast cancer specimens demonstrates marked variation similar to our previous observations in colon cancer. In summary, dUTPase represents an unexploited therapeutic target and the identification of effective inhibitors has the potential to improve the efficacy of 5-FU-based chemotherapies in a wide variety of cancers. To this end, a dUTPase pharmacophore model using in silico drug development techniques as a means to identify small molecule antagonists to dUTPase has been generated.

The above-mentioned and other features of this invention and the manner of obtaining and using them will become more apparent, and will be best understood, by reference to the following description, taken in conjunction with the accompanying drawings. The drawings depict only typical embodiments of the invention and do not therefore limit its scope.

### DESCRIPTION OF THE FIGURES

FIG. 1. Partial schematic diagram of thymidylate biosynthesis

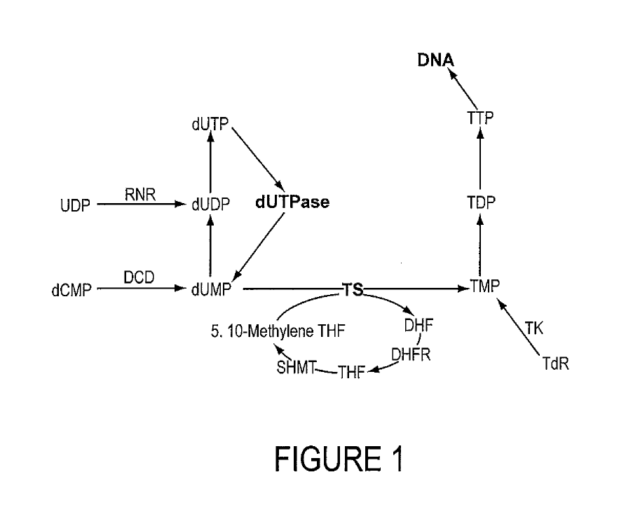

FIG. 2. Partial schematic diagram of thymidylate biosynthesis during thymidylate synthease (TS) inhibition.

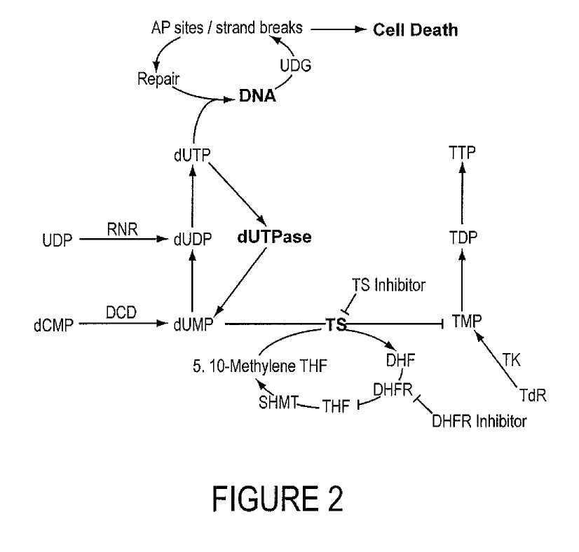

FIG. 3. Mechanism of 5-FU-induced DNA Damage. 5-Fluorouracil is converted to active metabolites: fluorodeoxyuridine monophosphate (FdUMP) and fluorodeoxyuridine triphosphate (FdUTP). FdUMP binds to, and inhibits the enzyme thymidylate synthase (TS) by formation of a ternary complex with the methyl donor co-factor. Inhibition of TS induces a metabolic blockade, resulting in depletion of thymidylate and the accumulation of dUMP which can be phosphorylated to dUTP. When dUTP pools expand, dUTPase activity can become saturated resulting in uracil misincorporation into DNA. In tumors with elevated expression of dUTPase, saturation of enzyme activity is unlikely to occur and dUTP misincorporation is eliminated as a mechanism of cytotoxicity. Generation of FdUTP and subsequent incorporation into DNA has also been demonstrated to induce significant cytotoxicity and dUTPase has been demonstrated to possess affinity for FdUTP, catalyzing its hydrolysis and preventing the misincorporation of FdUTP into DNA.

FIG. 4. Variation in dUTPase Expression in Cell Lines. Western blot analysis of dUTPase expression in a panel of colon (SW620, HCT-8, HCT116, HT29, LoVo) and gastric (AGS), mitochondrial and nuclear isoforms are indicated. β-actin was used to control for loading. dUTPase relative enzyme activity assay from corresponding Western blot cell lysates, Histogram bars represent the mean ±SEM of corresponding Western lysates analyzed in duplicate.

FIG. 5. Elevated dUTPase Protects Breast Cancer Cells from 5-FU. (A) Western blot and analysis of dUTPase expression and corresponding dUTPase enzyme activity assay following 72 h transfection with pTre-Tight:DUT-N in the presence and absence of 0.5 μg/m1 dox, Histogram bars represent the mean ±SEM of corresponding Western lysates analyzed in duplicate. (B) Analysis of dUTP accumulation in pTre-Tight:DUT-N-transfected MCF-7 psTet-off cells with basal (+dox) and induced (−dox) dUTPase expression and treated with 10 μM 5-FU, 1 μM FUdR and taxol (paclitaxel) 5 nM for 24 h, % dUTP was calculated as described in ‘Materials and Methods’, Histogram bars represent the mean ±SEM of two independent treatments, †=dUTP not detected. (C) Growth inhibition measured by MTS assay in pTre-Tight:DUT-N-transfected MCF-7 pTet-off cells following treatment with increasing concentrations (in μM) of 5-FU for 72 h. Histogram bars represent the mean ±SEM of three independent treatments, *=p<0.05, **=p<0.01.

FIG. 6. Immunohistochemical Analysis of dUTPase in Human Breast Adenocarcinoma. Formalin-fixed, paraffin-embedded breast cancer tumor specimens were routinely processed and stained using the DUT415 monoclonal antibody. A represents low dUTPase expression, B and C represent elevated nuclear and cytoplasmic expression. Photomicrographs A and B are 20× magnification, photomicrograph C is 40× magnification.

FIG. 7. Shape-merged pharmacophore model derived from Dud778. (A) Dud778 mapping onto the feature model. Green sphere represents the H-bond acceptor, and magenta represents H-bond donor. (B) Shape-merged feature model of Dud778 mapping onto the shape query. The gray area represents the shape constraint generated from the crystallized conformation. (C) Dud778 mapping onto the shape-merged feature model as shown in (B)

FIG. 8. Docking validation of Dud778-dUTPase co-crystal structure. (A) Dud778 as observed in the x-ray structure (1Q5H) is shown in green, and the atom-type colored conformation of Dud778 is predicted by GOLD. (B) superimposition of a representative compound, colored by atom type, with x-ray determined Dud778 conformation (green). The compound was selected from database screening that favorably interacts with the ligand binding domain by efficiently filling the deep cavity.

FIG. 9. Inhibitors of dUTPase induce growth arrest in the MCF-7 human breast cancer cell line. (A) MCF-7 cells were exposed to increasing concentrations of two novel dUTPase inhibitory lead compounds and growth inhibition was analyzed by MTS assay. The concentrations of compounds DU203 and BB-123 required to inhibit 50% cell growth was 65.1 and 73.8 μM.DU-203 enhances the growth arrest of MCF-7 cells when combined with FUdR. (B) DU-203 at a fixed concentration of 62.5 μM was combined with increasing concentrations of FUdR and the combined drug effect analyzed by MTS assay and the degree of synergy determined using the methods of Chou and Talalay. The combination of DU-203 and FUdR resulted in enhanced growth inhibition at all concentrations testec. BB-123 enhances the growth arrest of MCF-7 cells when combined with FUdR. (C) BB-123 at a fixed concentration of 62.5 μM was combined with increasing concentrations of FUdR and the combined drug effect analyzed as described previously. The combination of BB123 and FUdR resulted in enhanced growth inhibition at all concentrations tested.

FIG. 10. Schematic protocol for the development of receptor-based pharmacophore model. (A) 3-D structure of dUTPase (PDB code: 1q5h). Yellow and green represents β-sheet and purple represents α-helix. White is random coil. Cyan cycled area is the ligand binding pocket. (B) Amino acid residues in the binding pocket. (C) Complementaryfeatures mapped by H-bond donor probes (N—H groups represented by blue-white cylinders), H-bonds acceptor probes (O—H groups represented by red-white cylinder) and lipophilic probes (dots). (D) The optimized pharmacophore model. Each pair of magenta spheres represent the H-bond donor, each pair of green spheres represent H-bond acceptor. Cyan balls are hydrophobic sites.

### DETAILED DESCRIPTION OF THE INVENTION

dUTPase is a ubiquitous enzyme that is essential for viability in both prokaryotic and eukaryotic organisms (26, 48). As the main regulator of dUTP pools, the expression of dUTPase could have profound effects on the utility of chemotherapeutics that inhibit thymidylate biosynthesis and on various other diseases where dUTPase expression plays a role in disease.

The present invention relates to dUTPase inhibitors which are unique in characteristic as they are capable of inhibiting the activity of dUTPase and synergistically improving the efficacy of thymidylate synthease directed chemotherapy. These inhibitors promote dUTP accumulation and may be useful for the treatment of various diseases including but not limited to cancers, immune disorders including rheumatoid arthritis and inflammatory bowel disease as well as viral, parasitic, fungal, and bacterial infection.

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

### MATERIALS AND METHODS

Compounds and Reagents 5-fluorouracil (5-FU), fluorodeoxyuridine (FUdR) and taxol were purchased from Sigma (St. Louis, Mo.).

Cell Culture The human breast MCF-7 pTet-off cell line was obtained from BD Clontech (Mountainview, Calif.) and grown in DMEM supplemented with 10% tet-approved fetal bovine serum (BD Clontech) with penicillin/streptomycin and sodium pyruvate (Invitrogen Carlsbad, Calif.). Cells were maintained in a humidified Forma incubator (Thermoscientific, Waltham, Mass.) at 37° C. with 5% CO₂.

Overexpression of dUTPase MCF-7 pTet-off cells were seeded on 6 cm plates and 3 h after plating the cells were washed with PBS and fresh growth media added. After 24 h, cells were transfected with 2 μg pTre-Tight:DUT-N for 6 h, washed in PBS and the appropriate media added; to suppress the inducible expression of dUTPase, doxycycline (dox) was added to growth media containing tet-approved FBS at a final concentration of 0.5 μg/ml. Twenty-four h post-transfection, cells were plated for the appropriate assay and allowed to adhere for 24 h before media containing 5-FU, FUdR or taxol was added. Overexpression of dUTPase was confirmed using both Western blotting and enzyme activity assay.

dUTPase Activity Assay Cells were harvested and protein isolated and quantified as per Western blotting. Twenty-five μg of total protein was normalized to a 20 μl reaction volume with PBS/protease inhibitor. Relative dUTPase activity was determined as previously described (9) and is expressed as fold-change compared to an identical transfection in the presence of 0.5 μg/ml dox.

dUTP Accumulation Assay MCF-7 pTet-off cells were treated with specified concentrations of 5-FU, FUdR and taxol for indicated times, harvested, and 3×10⁶ cells were analyzed for nucleotide pool content using the assay developed by Sherman and Fyfe (10) modified to detect levels of TTP and dUTP by pre-incubating extracts with recombinant dUTPase (9, 11). Radioactive incorporation, measured in the presence of dUTPase represented the TTP pool, while untreated extracts represented both the dUTP and TTP pools. dUTP accumulation was determined by subtracting the results of extracts treated with dUTPase from untreated extracts and presented as % accumulation in histogram format. Statistical significance was determined using a two-tailed unpaired Student's t-test (Graphpad, San Diego, Calif.).

Antibodies and Western Blotting At specified time points, cells were collected and analyzed by Western blot as described previously (9). Western blots were probed overnight at 4° C. with affinity purified anti-dUTPase generated in our laboratory (1:500) and 2 h with appropriate secondary antibodies (goat-anti-mouse and goat-anti-rabbit HRP). Blots were re-probed for anti-β-actin (Sigma) to control for loading. HRP signal was detected using HyGlo and Hyblot film (Denville Scientific, Metuchen, N.J.) and developed on a Hope-Micromax film processor (Hope X-Ray, Warminster, Pa.).

Growth Inhibition Assay MCF-7 pTet-off cells were transfected in the presence or absence of 0.5 μg/ml of dox and growth inhibition was measured as previously (9) using CellTiter 96® AQueous One Solution (Promega, Madison, Wis.). Cells were exposed to increasing concentrations of 5-FU for 72 h. Absorbance was measured using a SpectraMax 190 microplate reader (Molecular Devices, Sunnyvale, Calif.) at 490 nm, with drug treated cells compared to untreated controls set at 100%. Statistical significance was determined using a two-tailed unpaired Student's t-test (Graphpad, San Diego, Calif.).

Immunohistochemistry (IHC) IHC using the DUT415 monoclonal antibody (2 μg/ml) was conducted on formalin-fixed, paraffin-embedded breast adenocarcinoma tissue samples using methods as previously described (12).

### RESULTS AND DISCUSSION

5-FU Mechanism of Action. Following entry into the cell, 5-FU is converted to its active metabolite, fluorodeoxyuridine monophosphate (FdUMP) whose primary mechanism of action is inhibition of thymidylate synthase (TS) by formation of a ternary complex with the methyl co-factor 5, 10-methylene tetrahydrofolate. This blocks the de novo synthesis of thymidylate resulting in perturbations in nucleotide pools, severe disruption of DNA synthesis and repair and ultimately leads to lethal DNA damage (1). Additional mechanisms of action include the incorporation of toxic fluoronucleotides into both DNA and RNA and the expansion of the intracellular deoxyuridine triphosphate (dUTP) pool and subsequent misincorporation into DNA (1). The 5-FU pro-drug capecitabine has the convenience of oral administration and has demonstrated equivalent efficacy as both a single agent and in combination with oxaliplatin for the first line treatment of mCRC (13, 14). Capecitabine is absorbed intact through the gastrointestinal mucosa where it undergoes a three-step enzymatic conversion to 5-FU exerting similar mechanisms of action (15).

Resistance to 5-FU. A significant number of studies have correlated intra-tumoral TS levels with response to fluoropyrimidine-based therapy (16-18). A study by Salonga et al. demonstrated for the first time that patients with CRC who responded to 5-FU therapy could be segregated by analysis of 3 genes involved in 5-FU metabolism; patients with low expression levels of TS, thymidine phosphorylase (TP) and dipyrimidine dehydrogenase (DPD) all responded to treatment and those with elevated expression in at least one of the three genes did not (18). A meta-analysis performed by Popat et al. analyzed 20 independent studies consisting of over 3000 patients and concluded that those with elevated TS expression demonstrated poorer overall survival compared with tumors expressing low levels (19). Functional genomic polymorphisms have been identified within the 5′-region and the 3′-UTR of the TS gene and have been associated with response to fluoropyrimidine treatment through TS mRNA transcription and stability (20-23). Although conflicting results have been reported in the literature (24), higher TS protein and mRNA expression in tumors has generally been associated with poor clinical outcome in patients treated with 5-FU-based chemotherapy (19).

Additional enzymes involved in 5-FU metabolism have been associated with response to treatment. DPD catalyses the rate-limiting step in the catabolism of 5-FU with >80% of 5-FU degraded by DPD in the liver. Variation in DPD expression therefore has direct impact on 5-FU bioavailability (25). A previously mentioned study by Salonga et al. demonstrated that CRC patients who responded to 5-FU therapy had low expression levels of TS, TP and DPD and non-responders had elevated expression in at least one of the three genes (18).

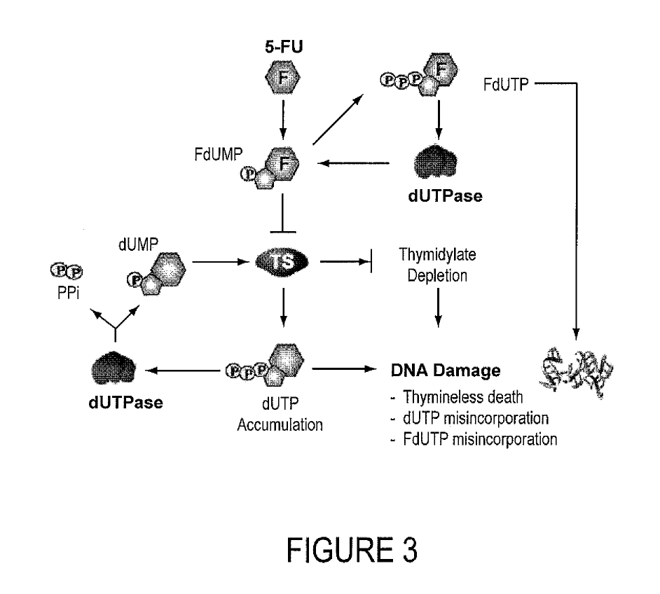

The Role of dUTPase in 5-FU Resistance. Deoxyuridine triphosphate nucleotidohydrolase (dUTPase) is the sole enzyme responsible for the hydrolysis of dUTP to dUMP and pyrophosphate, providing substrate for thymidylate synthase (TS) and eliminating dUTP from the DNA biosynthetic pathway. Although dUTP is a normal intermediate in DNA synthesis, the extensive expansion of the dUTP pool and subsequent uracil misincorporation into DNA is lethal in both prokaryotic and eukaryotic organisms as demonstrated from knockout models (26). Importantly, uracil misincorporation is a significant mechanism of cytotoxicity induced by TS-inhibiting chemotherapeutic agents including 5-FU and FUdR (1, 27, 28). Expression of dUTPase is therefore reported to be an important determinant of cytotoxicity induced by agents that target TS both in vitro and in vivo. Overexpression of dUTPase is reported to abrogate the expansion of intracellular dUTP following TS-inhibition, providing substrate for TS in the form of clUMP and preventing DNA damage associated with uracil misincorporation. In addition, dUTPase is reported to target FdUTP for catalysis, thus preventing the accumulation of DNA damage reported to occur following FdUTP misincorporation into DNA (29-31) (FIG. 3).

We previously reported that diminished dUTPase expression enhanced dUTP pool expansion following treatment with a TS-inhibitor, sensitizing yeast cells to the effects of uracil misincorporation while cells overexpressing dUTPase were significantly resistant (32). In colon cancer cells, induced expression of dUTPase was reported to confer resistance to FUdR (33) while our laboratory has previously demonstrated that silencing dUTPase expression using siRNA sensitized both breast and colon cell lines to FUdR through, dUTP pool expansion, uracil misincorporation and enhanced DNA fragmentation (9). Moreover, we also reported the results of a small retrospective clinical study negatively correlating elevated nuclear expression of dUTPase with response to 5-FU-therapy in CRC patients, bolstering the concept that dUTPase represents an attractive drug target (12). The FOCUS clinical trial analyzed dUTPase expression (High vs Low) in 846 advanced CRC patients randomized to 5-FU alone, 5-FU+oxaliplatin, or 5-FU+irinotecan and demonstrated a negative association between elevated dUTPase and response to therapy and time to tumor progression (34). Validation in an independent 486 patient follow-up analysis demonstrated that dUTPase retained an association with time to tumor progression (35).

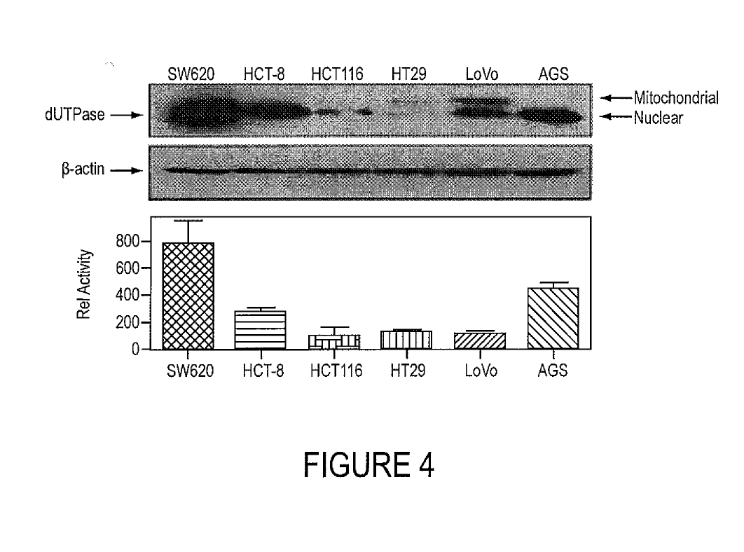

Dysregulation and variation in expression of dUTPase is observed in many cancer cell lines (FIG. 4) and tumor specimens including tumor types frequently treated with agents that target TS, thus validation of dUTPase expression as a marker of resistance to TS-directed chemotherapy such as 5-FU is of clinical interest (12, 28, 36).

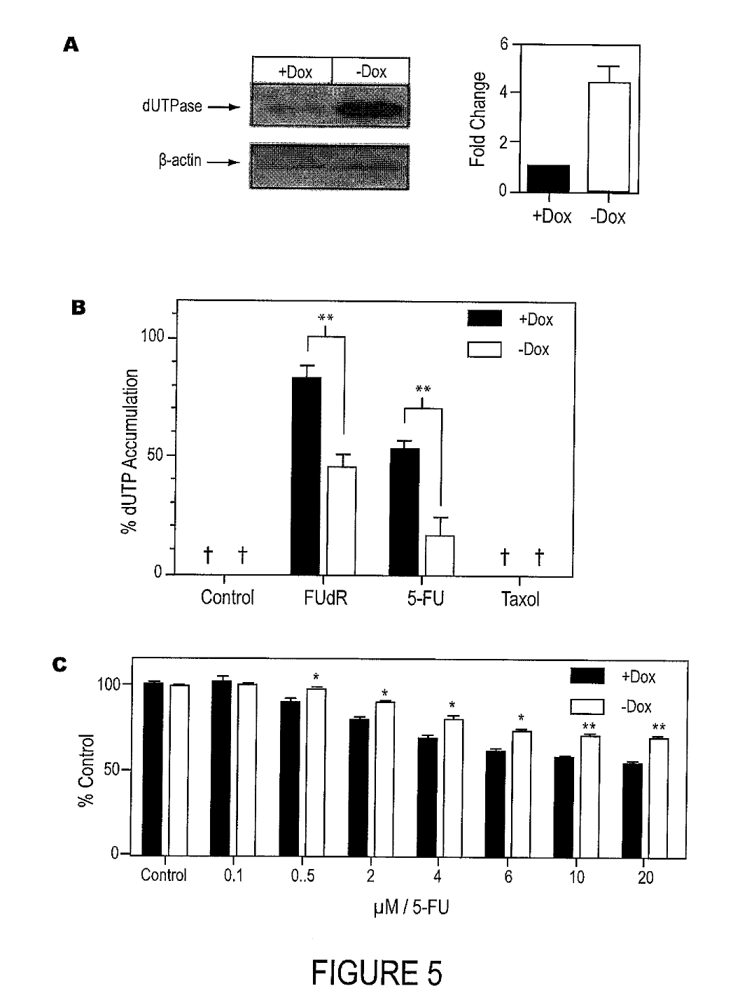

Elevated dUTPase Protects Breast Cancer Cells from 5-FU. Our laboratory has previously demonstrated in the MCF-7 breast cancer model, that diminishing dUTPase expression resulted in a significant increase in dUTP pools and enhanced cytotoxicity induced by the fluoropyrimidine FUdR. As an extension to this, we sought to determine if overexpression of dUTPase could significantly protect MCF-7 cells from the expansion of dUTP pools and reduce the growth inhibitory effects of 5-FU. A tetracycline-repressible dUTPase expression construct and overexpressed dUTPase with an approximate 4-fold increase in expression and enzymatic activity in transfected tet-inducible MCF-7 pTet-off cells (FIG. 5A) was utilized. We demonstrate that cells which overexpress dUTPase were significantly more resistant to 5-FU-induced dUTP pool expansion with a 37% reduction in dUTP detected compared to control transfected cells with basal dUTPase. We also utilized FUdR, a more TS-directed fluoropyrimidine and observed a similar 38% reduction in dUTP pool expansion in the presence of increased dUTPase expression when compared to basal expression. As a control we used paclitaxel, which inhibits cell growth by binding to dynamic microtubules (37) and demonstrate that treatment with a non-TS-directed agent resulted in no detection of dUTP in the presence of basal or induced dUTPase expression (FIG. 5B).

We subsequently investigated the effects of dUTPase overexpression on growth inhibition following exposure of MCF-7 pTet-off cells to increasing concentrations of 5-FU. Despite the inherent-resistance of MCF-7 cells to 5-FU (>10 μM IC₅₀), overexpression of dUTPase resulted in a statistically significant decrease in growth inhibition compared to cells expressing basal dUTPase at all concentrations of 5-FU tested (FIG. 5C). These data confirm and extend our previous observations in MCF-7 parental cells, where depletion of dUTPase correlated with increased dUTP pool expansion, greater DNA double strand break formation, and enhanced chemosensitivity to FUdR. These data further validate the significance of the uracil-DNA pathway as a mechanism of resistance to 5-FU-based therapies in breast cancer.

Fluoropyrimidine-based Strategies in Breast Cancer. 5-FU has proven itself as the mainstay agent in therapeutic combinations used to treat CRC. More recently, the 5-FU pro-drug capecitabine has demonstrated efficacious combinations with agents approved for a variety of different cancers and has recently been of particular interest in the treatment of chemorefractory breast cancer. A recent clinical trial demonstrated improved efficacy when capecitabine was used in combination with the microtubule-stabilizing agent ixabepilone when compared to capecitabine alone in heavily pre-treated metastatic breast cancer patients (8). In a HER2-expressing patient population, capecitabine in combination with the dual EGFR/HER2 tyrosine kinase inhibitor lapatinib, demonstrated improved progression free survival in heavily pre-treated metastatic breast cancer patients (7). These clinical trials demonstrate that patients who previously failed multiple lines of therapy which included anthracyclines and taxanes still derive benefit from a fluoropyrimidine-based therapy. While only preliminary evidence exists regarding resistance to capecitabine, the distinct overlap in mechanism of action with 5-FU would imply that similar mechanisms of resistance should exist for both agents and as a result, dUTPase expression should be investigated as a marker of response.

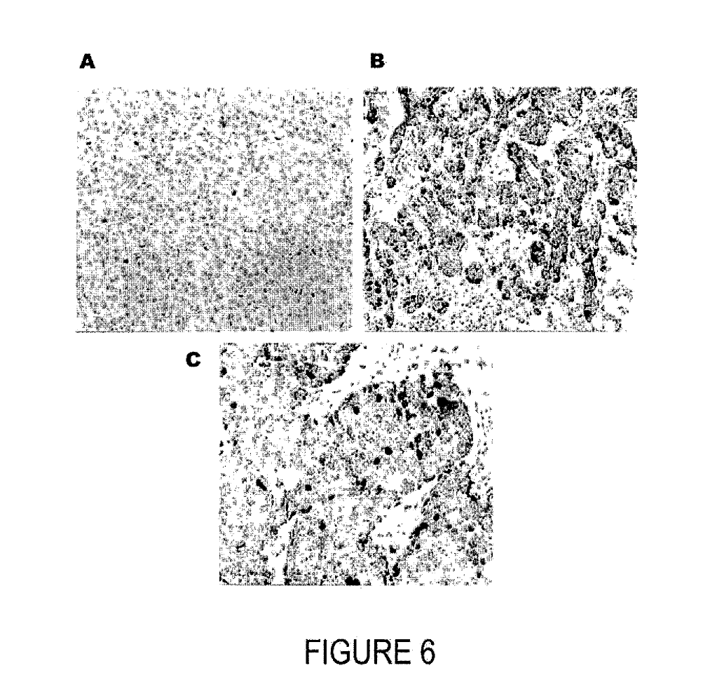

Dysregulation of dUTPase Expression in Breast Adenocarcinoma. Previously, we performed IHC analyzing dUTPase expression and localization in colon cancer specimens from patients treated with 5-FU-based therapy. This study negatively correlated elevated expression of intratumoral dUTPase with response to 5-FU therapy (12). We therefore performed IHC on breast adenocarcinoma tissue using the DUT415 monoclonal antibody to investigate both dUTPase expression and intracellular localization. Interestingly, similar to our previous observations in colon cancer specimens, we report that dUTPase demonstrates significant variation in total expression and localization. FIG. 6 shows three representative photomicrographs obtained following IHC analysis and demonstrating breast adenocarcinomas with low expression (A), and elevated nuclear and cytoplasmic expression of dUTPase (B and C). This study demonstrates that the previously reported variation in dUTPase expression in colon cancer specimens extends to other neoplastic tissues including breast cancer, in which fluoropyrimidine-based therapies are routinely implemented.

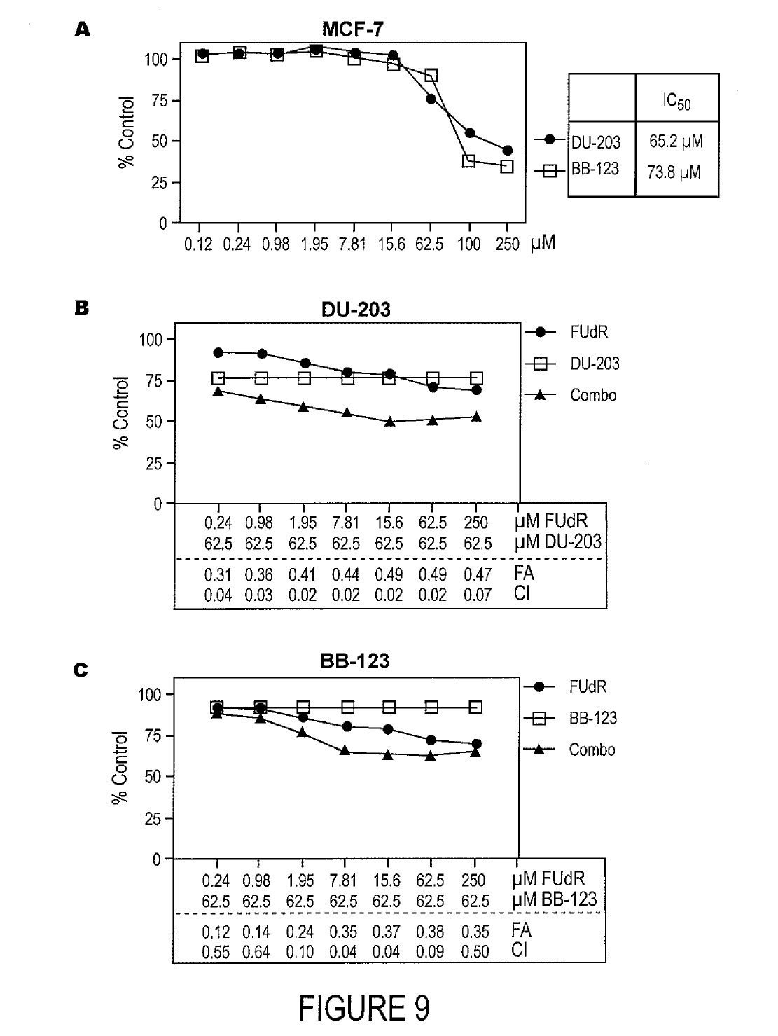

Inhibitors of dUTPase induce growth arrest in the MCF-7 human breast cancer cell line. DU-203 and BB-123 have demonstrated growth inhibitory capabilities in the MCF-7 human breast cancer cell line. These growth inhibitory effects are consistent with the inhibition of dUTPase and the resultant accumulation of dUTP and uracil misincorporation during DNA replication. To test the specificity of these inhibitors, DU-203 and BB-123 were combined with the inhibitor of thymidylate synthase (TS), FUdR. We have previously demonstrated that the MCF-7 can be sensitized to the effects of FUdR when dUTPase expression is depleted by RNA interference. When DU-203 and BB-123 are combined with FUdR, enhanced growth inhibition is observed at all concentrations tested (FIG. 9). This would strongly suggest that concomitant inhibition of TS and dUTPase is resulting in the accumulation of dUTP and an increase in cytotoxicity as a result of uracil misincorporation during the DNA repair process.

In Silico Identification of dUTPase Small Molecule Antagonists. Evidence suggests that the accumulation of excessive dUTP pools and its iterative misincorporation into DNA may be a key mediator of cytotoxicity subsequent to inhibition of de nouo thymidylate biosynthesis. As the sole regulator of dUTP pools, dUTPase represents an intervention point that could improve the clinical efficacy of this important class of TS-targeted chemotherapeutic agents. In addition to the pathway driven rationale for dUTPase as a therapeutic target, several other practical advantages make this enzyme amenable to drug development: 1) dUTPase has a well characterized, non-redundant cellular function; 2) crystallographic data of enzyme-substrate complexes are available at high resolution (38); 3) the biochemical consequences of dUTPase inhibition have measurable outcomes that can be used to validate the mechanism of drug action.

The in silico approach to inhibitor design is a useful technology that utilizes existing structural data from crystallographic and site-directed mutagenesis studies to identify lead inhibitory compounds with optimum potency, selectivity and/or pharmacokinetic properties. Pharmacophore models are used as queries to cull structurally diverse molecules that may potentially show requisite inhibitory activity from existing chemical databases. Such an approach allows for rational design of inhibitory molecules to a therapeutic target by enabling establishment of quantitative-structure activity relationships between potential molecules and their inhibitory ability (39).

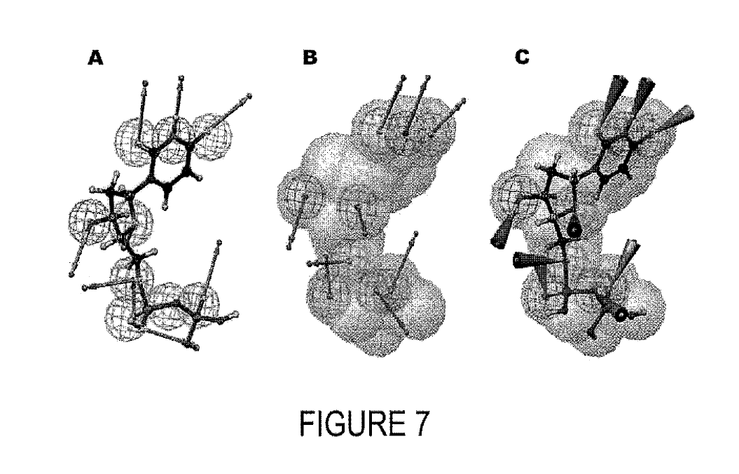

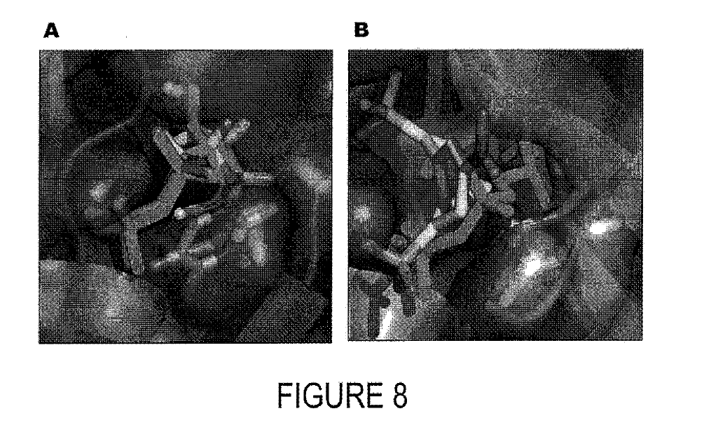

To identify potential inhibitors to human dUTPase, the Structure-Based Focusing (SBF) module equipped in the Cerius2 software package (Accelrys, Inc.) was used to efficiently generate a pharmacophore model by mapping the functional features of the dUTPase active site from the crystal structure (1Q5H) of recombinant human dUTPase enzyme bound to a dUDP substrate (Dud778). We defined the binding site centered at the average position of Dud778, a ligand co-crystallized with human dUTPase in the x-ray structure, and with a radius of 15 Å covering the ligand binding cavity. Catalyst software (Accelrys, Inc.) package was employed to map the functional features (H-bond donor, H-bond acceptor, hydrophobic feature, or aromatic ring) onto the ligand (Dud778) according to the active confirmation observed in the x-ray structures. To develop the feature model, geometrical constraints were assigned to each feature, and the selected features were merged to a single model (FIG. 7). The resulting pharmacophore model was then queried against our in-house compound database for potential dUTPase antagonists (potential antagonist may be purchased from Asinex, Inc.). Docking simulation approaches have proven useful in accurately predicting small molecule binding interactions with a receptor. To further refine selection of compounds for testing in our in vitro enzymatic assay, we utilized GOLD (Genetic Optimization for Ligand Docking, Version 2.1) software to perform docking studies on our 24-processor Silicon Graphics Onyx workstation (40) and ranked the candidate antagonists according to the predicted docking scores. As a result, those candidate molecules displaying inefficient contact with the dUTPase active site were not tested in the enzyme assay. The most favorable docking conformation was nearly identical to the x-ray structure indicating that surface docking is an efficient approach to sampling the ligand-protein interactions in the dUTPase system (FIG. 8). The binding pocket is characterized by a deep cavity localized by the uracil base motif and a channel filled by phosphate fragment. As a final selection step, ADMETPredictor (Simulations Plus, Inc., Lancaster, Calif.) software was used to calculate the ADMET (absorption, distribution, metabolism, excretion and toxicity) properties of candidate molecules.

The pharmacophore-based search of a database of approximately 360,000 small molecules yielded an initial set of 400 structurally diverse compounds possessing chug-like physicochemical properties. These molecules were tested in our dUTPase activity assay at an initial 100 μM in vitro to determine their percentage inhibition of dUTPase catalysis relative to un-inhibited control. Table 1 illustrates the docking fitness scores of the top four active compounds and their ability to inhibit dUTPase activity. Ranked according to inhibitory activity, two representative inactive molecules are also shown with their corresponding docking scores, indicating a reasonable correlation between predicted docking fitness values and the dUTPase inhibitory potential of these small molecules. Lead compounds (Table 2) with promising inhibitory activity can be further optimized in structure-activity studies in to develop more potent analogs in vitro that should also demonstrate clinical utility as dUTPase inhibitors in cellular cancer disease models.

The Future of 5-FU. Fifty-one years since Charles Heidelberger first described the anti-tumor activity of the fluoropyrimidines, many tens of thousands of cancer patients worldwide have benefited from chemotherapy stemming from those original observations (41). 5-FU and other fluoropyrimidine derivatives continue to demonstrate clinical efficacy in a wide variety of cancers and remain central agents in all treatment options in CRC and an effective agent in refractory breast cancer patients. However, a large number of patients do not respond to 5-FU-based treatment strategies and progress remains hindered by the lack of predictive markers to these therapies. Therefore, identification and exploitation of alternative targets within this pathway of proven therapeutic utility is of considerable importance. As cancer treatment undergoes a paradigm shift toward individualized treatment strategies, the identification and exploitation of targets such as dUTPase should deliver a more effective 5-FU-based chemotherapy, increasing response rates in advanced and metastatic disease and enabling more patients to undergo complete surgical resection with curative intent.

TABLE 1
Compounds Identified using the dUTPase Pharmacophore Model.

| | GOLD Docking | Molecular | % dUTPase |
|---|---|---|---|
| Compound ID | Score^{†} | Weight | Inhibition^{‡} |
| DU7 | 70.4 | 443.52 | 55.72 |
| DU6 | 64.5 | 523.99 | 46.24 |
| BB37 | 66.4 | 418.5 | 33.98 |
| DU44 | 78.9 | 680.86 | 28.46 |
| DU202 | 56.6 | 494.54 | Inactive |
| DU53 | 56.5 | 535.68 | Inactive |

^{†}Docking score calculated using GOLD software docking simulations.
^{‡}Percentage inhibition of dUTPase enzymatic catalysis by respective compound at a fixed concentration of 100 μM. Activity assay was performed as described in ‘Materials and Methods.’

TABLE 2
Lead compounds and associated activity

| | Compound ID | Structure |
|---|---|---|
| 1 | G11P44 MW = 315.34 (Activity = 79.40%) | 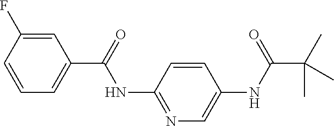 |
| 2 | E6P39 MW = 424.54 (Activity = 45.93%) | 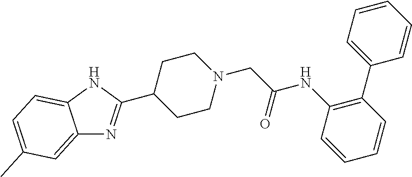 |
| 3 | A2P45 MW = 336.32 (Activity = 45.19%) | 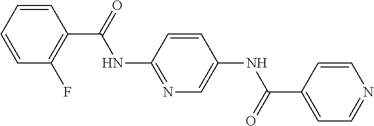 |
| 4 | C7P10 MW = 405.49 (Activity = 45.01%) | 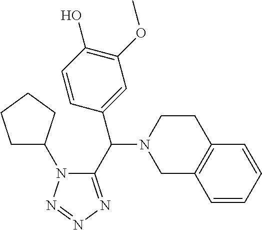 |
| 6 | E7P39 MW = 356.4 (Activity = 43.95%) | 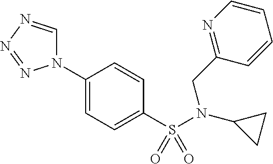 |
| 7 | E7P41 MW = 398.54 (Activity = 45.24%) | 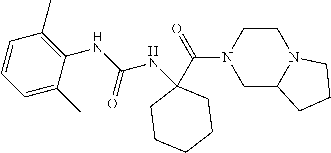 |
| 8 | G2P7 MW = 355.45 (Activity = 41.63%) | 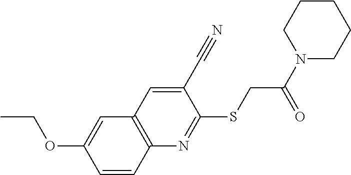 |
| 9 | E7P36 MW = 347.39 (Activity = 41.61%) | 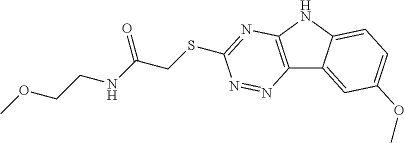 |
| 10 | D3P25 MW = 327.44 (Activity = 40.22%) | 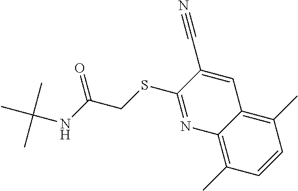 |
| 11 | E2P34 MW = 427.5 (Activity = 39.82%) | 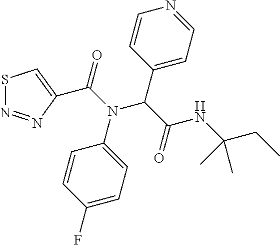 |
| 12 | E5P44 MW = 361.46 (Activity = 39.57%) | 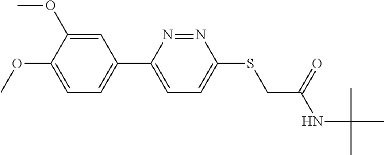 |
| 13 | E8P41 MW = 333.43 (Activity = 39.22%) | 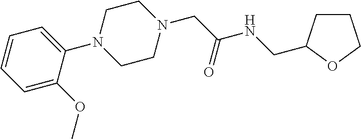 |
| 14 | E7P32 MW = 404.91 (Activity = 39.57%) | 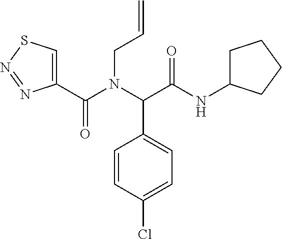 |
| 15 | E6P44 MW = 442.58 (Activity = 38.79%) | 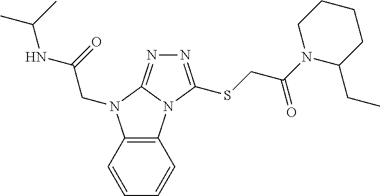 |
| 16 | C10P43 MW = 419.46 (Activity = 38.5%) | 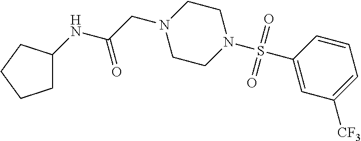 |
| 17 | F5P35 MW = 276.36 (Activity = 38.03%) | 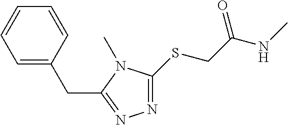 |
| 18 | D3P24 MW = 424.53 (Activity = 37.04%) | 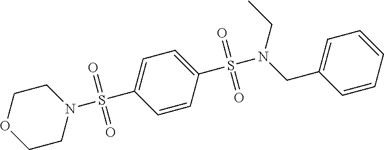 |
| 19 | C11P25 MW = 328.47 (Activity = 36.95%) | 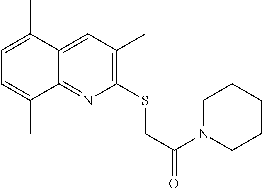 |
| 20 | Du 43 MW = 495.94 (Activity = 46.30%) | 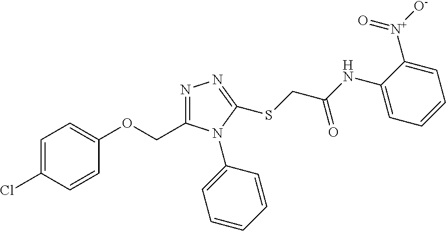 |
| 21 | Du 42 MW = 542.62 (Activity = 40.21%) | 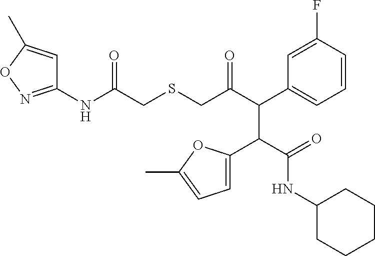 |
| 22 | Du 202 MW = 494.54 (Activity = 38.78%) | 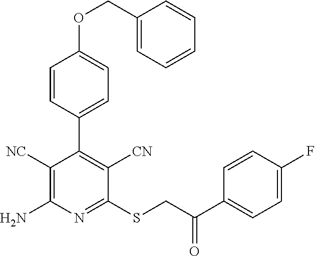 |
| 23 | Du 201 MW = 494.99 (Activity = 38.70%) | 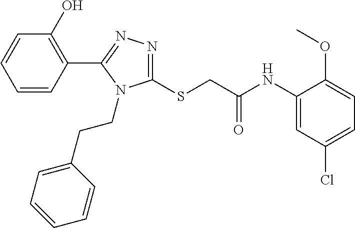 |
| 24 | Du 200 MW = 433.5 (Activity = 38.54%) | 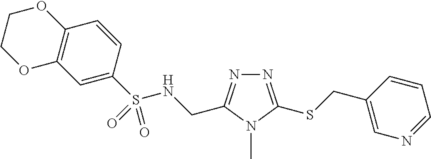 |
| 25 | Du 47 MW = 445.56 (Activity = 38.18%) | 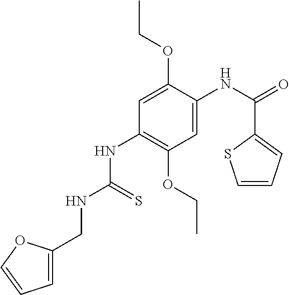 |
| 26 | Du 203 MW = 531.52 (Activity = 25.67%) | 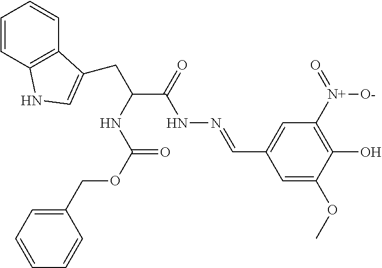 |
| 27 | Du 49 MW = 452.55 (Activity = 32.07%) | 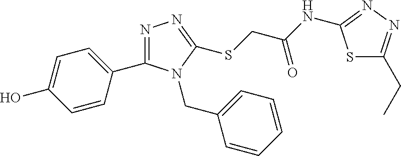 |
| 28 | BB123 MW = 399.57 (Activity = 27.51%) | 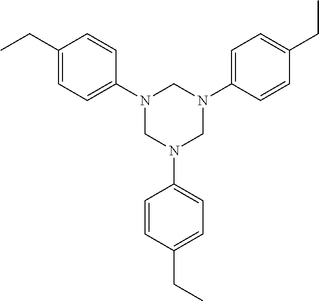 |
| 29 | BB37 MW = 418.47 (Activity = 28.70%) | 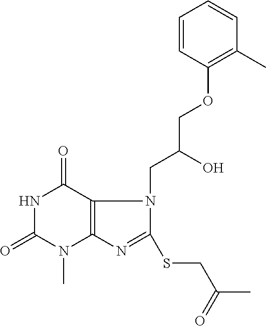 |
| 30 | Du 6 MW = 523.99 (Activity = 31.84%) | 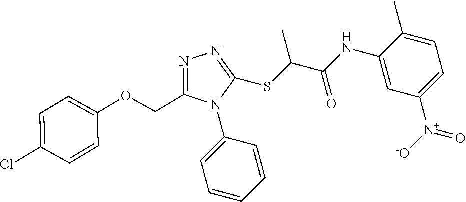 |
| 31 | Du 7 MW = 443.52 (Activity = 35.24%) | 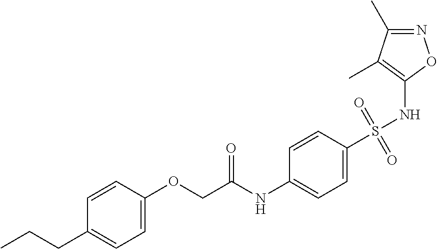 |
| 32 | Du 44 MW = 680.09 (Activity = 40%) | 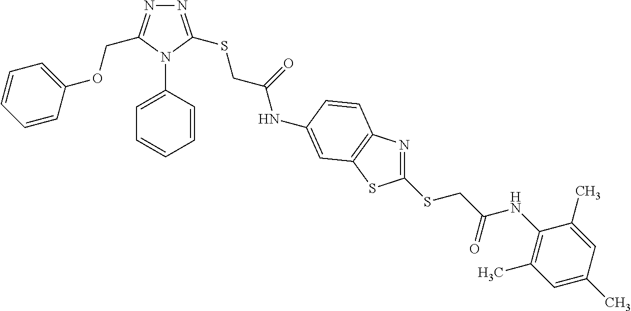 |
| 33 | Du 16 MW = 457.52 (Activity = 30.91%) | 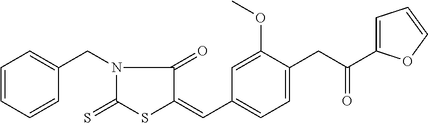 |
| | Compound ID | Claims/Formulas |
| 1 | G11P44 MW = 315.34 (Activity = 79.40%) | 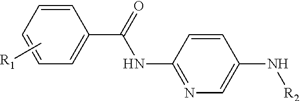 |
| 2 | E6P39 MW = 424.54 (Activity = 45.93%) | 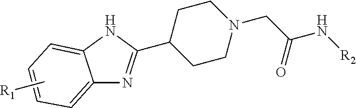 |
| 3 | A2P45 MW = 336.32 (Activity = 45.19%) | 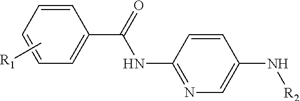 |
| 4 | C7P10 MW = 405.49 (Activity = 45.01%) | 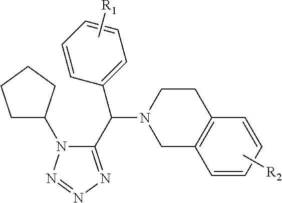 |
| 6 | E7P39 MW = 356.4 (Activity = 43.95%) | 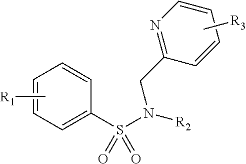 |
| 7 | E7P41 MW = 398.54 (Activity = 45.24%) | 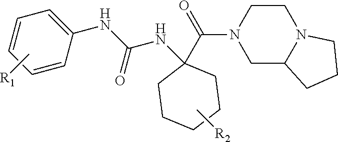 |
| 8 | G2P7 MW = 355.45 (Activity = 41.63%) | 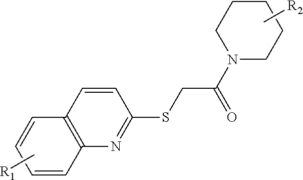 |
| 9 | E7P36 MW = 347.39 (Activity = 41.61%) | 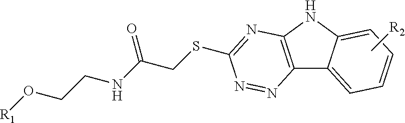 |
| 10 | D3P25 MW = 327.44 (Activity = 40.22%) | 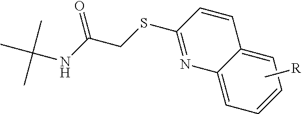 |
| 11 | E2P34 MW = 427.5 (Activity = 39.82%) | 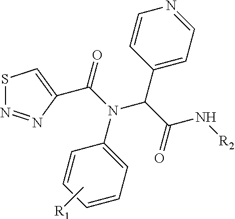 |
| 12 | E5P44 MW = 361.46 (Activity = 39.57%) | 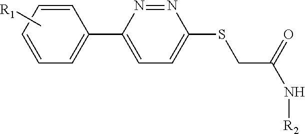 |
| 13 | E8P41 MW = 333.43 (Activity = 39.22%) | 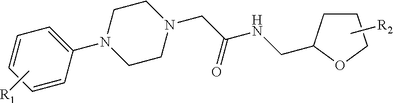 |
| 14 | E7P32 MW = 404.91 (Activity = 39.57%) | 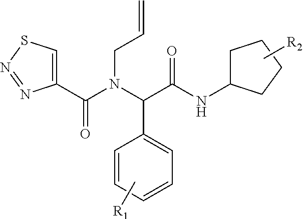 |
| 15 | E6P44 MW = 442.58 (Activity = 38.79%) | 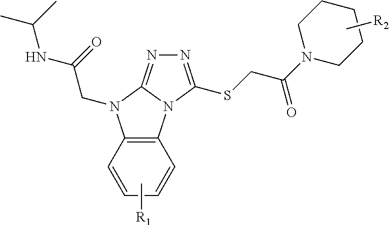 |
| 16 | C10P43 MW = 419.46 (Activity = 38.5%) | 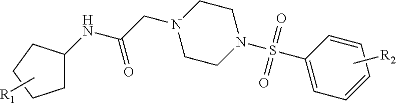 |
| 17 | F5P35 MW = 276.36 (Activity = 38.03%) | 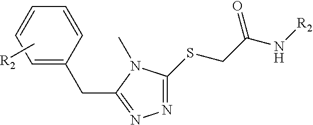 |
| 18 | D3P24 MW = 424.53 (Activity = 37.04%) | 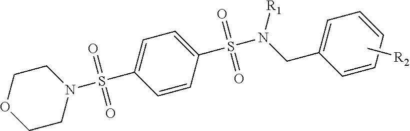 |
| 19 | C11P25 MW = 328.47 (Activity = 36.95%) | 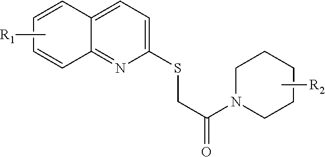 |
| 20 | Du 43 MW = 495.94 (Activity = 46.30%) | 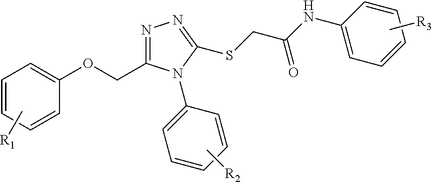 |
| 21 | Du 42 MW = 542.62 (Activity = 40.21%) | 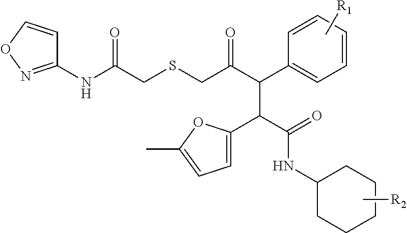 |
| 22 | Du 202 MW = 494.54 (Activity = 38.78%) | 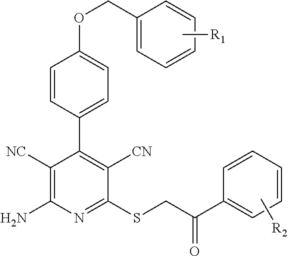 |
| 23 | Du 201 MW = 494.99 (Activity = 38.70%) | 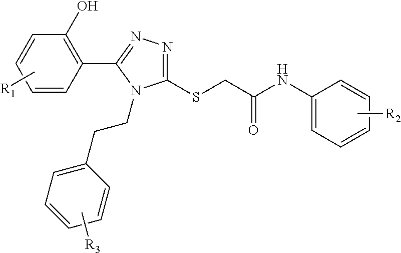 |
| 24 | Du 200 MW = 433.5 (Activity = 38.54%) | 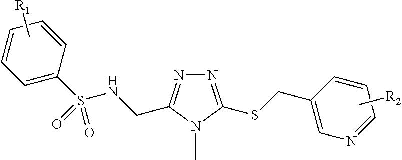 |
| 25 | Du 47 MW = 445.56 (Activity = 38.18%) | 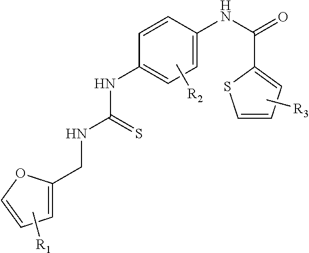 |
| 26 | Du 203 MW = 531.52 (Activity = 25.67%) | 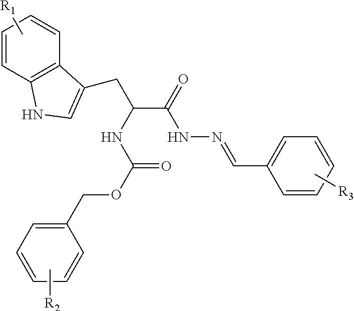 |
| 27 | Du 49 MW = 452.55 (Activity = 32.07%) | 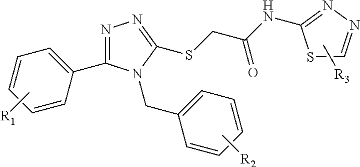 |
| 28 | BB123 MW = 399.57 (Activity = 27.51%) | 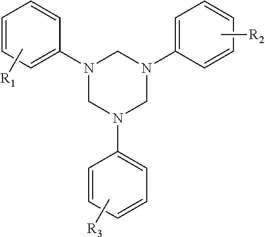 |
| 29 | BB37 MW = 418.47 (Activity = 28.70%) | 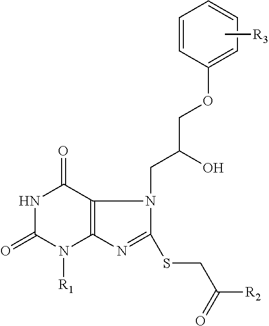 |
| 30 | Du 6 MW = 523.99 (Activity = 31.84%) | 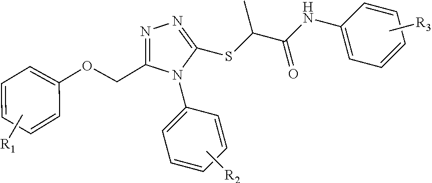 |
| 31 | Du 7 MW = 443.52 (Activity = 35.24%) | 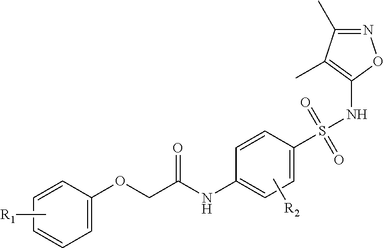 |
| 32 | Du 44 MW = 680.09 (Activity = 40%) | 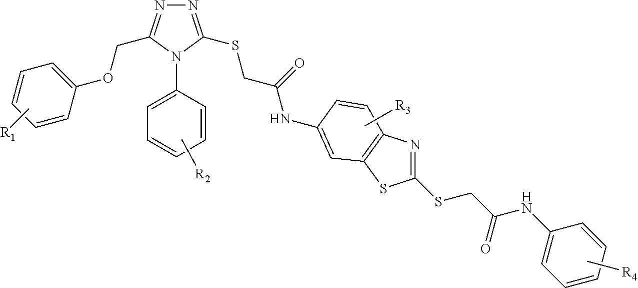 |
| 33 | Du 16 MW = 457.52 (Activity = 30.91%) | 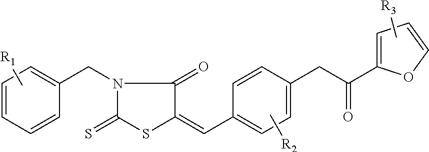 |

R₁-R₄ groups in Formulas 1-32 are defined as: R₁-R₄ taken independently or together are a hydrogen atom, a halogen atom, a hydroxyl group, or any other organic groups containing any number of carbon atoms, preferably 1-20 carbon atoms, and optionally include a heteroatom such as oxygen, sulfur, or nitrogen in a linear, branched or cyclic structural formats. Representative R₁-R₃ groups include (not limited to) alkyl, substituted alkyl alkenyl, substituted alkenyl, alkynyl substituted, phenyl, substituted phenyl, aryl, substituted aryl, heteroaryl, substituted heteroaryl. Representative substitutions include (not limited to) halo, hydroxyl, alkoxy, alkylthio, phenoxy, aroxy, cyano, isocyano, carbonyl, carboxyl, amino, amido, sulfonyl and substituted heterocyclics.

### EXAMPLES

Development of Novel Structure-based Drug Discovery Models. To begin to rationally identify potential inhibitors to human dUTPase, we utilized the crystal structure of recombinant human dUTPase enzyme bound to substrate (Dud778). Three drug selection models were generated to begin this analysis. One of the ligand itself (ligand-based model), the ligand binding pocket within the dUTPase trimetric structure (receptor-based model) and structure-based docking studies to identify candidate compounds (docking model).

Ligand-based Pharmacophore Model. The pharmacophore model developed from the Dud778, the ligand that was co-crystalized with human dUTPase. The shape-merged model of Dud778 (FIG. 7c) was applied to screen a representative selection of 150,000 compounds from our in-house database. Overall nearly 1,500 compounds were identified by the shape-merged models.

Structure-based pharmacophore model. Characterizing the binding pocket of a target structure is a critical step in identifying novel inhibitors with the complementary features in the target receptor binding pocket. This approach has been successfully applied to several drug targets including our previous work with drug discovery against HIV-1 integrase. We propose to apply the approach in the dUTPase inhibitor design.

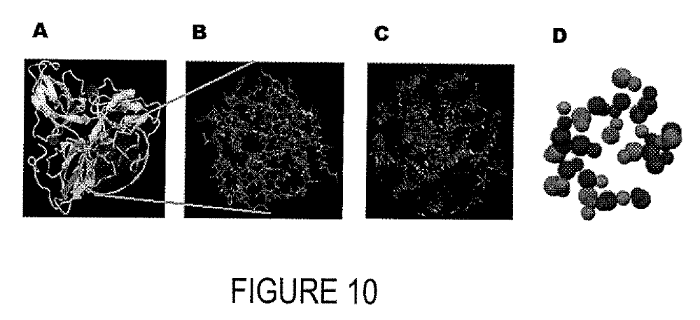

To identify potential inhibitors to human dUTPase, the Structure-Based Focusing (SBF) module equipped in the Cerius2 software package (Accelrys, Inc.) was used to efficiently generate a pharmacophore model by mapping the functional features of the dUTPase active site from the crystal structure (1Q5H) of recombinant human dUTPase enzyme bound to a dUDP substrate (Dud778). We defined the binding site centered at the average position of Dud778, a ligand co-crystallized with human dUTPase in the x-ray structure, and with a radius of 15 Å covering the ligand binding cavity (FIG. 10a, 10b). Catalyst software (Accelrys, Inc.) package was employed to map the functional features (H-bond donor, H-bond acceptor, hydrophobic feature, or aromatic ring) onto the ligand (Dud778) according to the active confirmation observed in the x-ray structures (FIG. 10c). To develop the feature model, geometrical constraints were assigned to each feature, and the selected features were merged to a single pharmacophore model (FIG. 10d). The resulting pharmacophore model was then queried against our in-house compound database for potential selective dUTPase antagonists.

Structure-based pharmacophore model. Characterizing the binding pocket of a target structure is a critical step in identifying novel inhibitors with the complementary features in the target receptor binding pocket. This approach has been successfully applied to several drug targets including our previous work with drug discovery against HIV-1 integrase. We propose to apply the approach in the dUTPase inhibitor design.

To identify potential inhibitors to human dUTPase, the Structure-Based Focusing (SBF) module equipped in the Cerius2 software package (Accelrys, Inc.) was used to efficiently generate a pharmacophore model by mapping the functional features of the dUTPase active site from the crystal structure (1Q5H) of recombinant human dUTPase enzyme bound to a dUDP substrate (Dud778). We defined the binding site centered at the average position of Dud778, a ligand co-crystallized with human dUTPase in the x-ray structure, and with a radius of 15 Å covering the ligand binding cavity (FIG. 10a, 10b). Catalyst software (Accelrys, Inc.) package was employed to map the functional features (H-bond donor, H-bond acceptor, hydrophobic feature, or aromatic ring) onto the ligand (Dud778) according to the active confirmation observed in the x-ray structures (FIG. 10c). To develop the feature model, geometrical constraints were assigned to each feature, and the selected features were merged to a single pharmacophore model (FIG. 10d). The resulting pharmacophore model was then queried against our in-house compound database for potential selective dUTPase antagonists.

Structure-Based Docking Model—Systematic Selection of Representative Hit Molecules Identified by Pharmacophore Models.

No previous docking studies are reported using human dUTPase crystal structures. Our first goal was to validate the Dud778 binding mode predicted by docking study. Our most favorable docking conformation was nearly identical to the x-ray structure (see FIG. 8a), indicating surface docking is an efficient approach to sampling the ligand-protein interactions in the dUTPase system. The binding pocket is characterized by a deep cavity localized by the uracil base motif and a channel filled by phosphate fragment. The docking scores of the searched compounds were in the range of −550.23 to +97.94, with higher scores representing better docking values. Visualization of the docking complex showed that the poor docking scores had bad contact between the ligand and the receptor, unfavorable internal conformation, or the inefficient pocket usage. A top-scored compound binding conformation as predicted from docking simulations is superimposed with Dud778 (FIG. 8b). The top compounds with docking score over 40.0 were selected for ranking.

Docking simulation approaches have proven useful in accurately predicting small molecule binding interactions with a receptor. To further refine selection of compounds for testing in our in vitro enzymatic assay, we utilized GOLD (Genetic Optimization for Ligand Docking, Version 2.1) software to perform docking studies on our 24-processor Silicon Graphics Onyx workstation (40) and ranked the candidate antagonists according to the predicted docking scores. As a result, those candidate molecules displaying inefficient contact with the dUTPase active site were not tested in the enzyme assay. As a final selection step, ADMETPredictor (Simulations Plus, Inc., Lancaster, Calif.) software was used to calculate the ADMET (absorption, distribution, metabolism, excretion and toxicity) properties of candidate molecules. The pharmacophore-based search of a database of approximately 5,000,000 small molecules yielded structurally diverse compounds possessing drug-like physicochemical properties. To begin, 6,000 of the top ranked compounds possessing the best docking fitness scores were acquired for testing in subsequent in vitro screening assays.

Screening of candidate compounds. Six thousand of the top candidate compounds were systematically screened for dUTPase inhibitory activity using two assays: 1) a medium throughput in vitro assay, adapted in our laboratory, that detects the release of pyrophosphate from the dUTPase reaction (PiPer-dUTPase assay) and 2) a well established, thin layer chromatography-based radioactive assay that detects the release of H³-dUMP from the dUTPase reaction (radioactive-dUTPase assay) (11). The PiPer-dUTPase assay was used as an initial medium throughput screen to rapidly evaluate compounds (˜500 compounds tested per week) and those compounds that demonstrated significant inhibitory activity were subsequently cross validated utilizing the radioactive-dUTPase assay (˜50 compounds per week).

The medium throughput PiPer-dUTPase was adapted from the Molecular Probes PiPer Pyrophosphate Kit that provides a sensitive method to detect free pyrophosphate in solution through the formation of the fluorescent product resorufin. This approach has been used to monitor enzyme kinetics of pyrophosphate releasing enzymes including: DNA and RNA polymerases and adenylate cyclase. This assay was adapted by simply utilizing the dUTPase reaction as a source of pyrophosphate for the PiPer reaction. Recombinant dUTPase and dUTP were added with the candidate inhibitors and monitored for inhibitory activity. In each case, the inhibitors were also counter-screened with pyrophosphate alone (no dUTPase added) to ensure that they did not interfere with the kinetics of the PiPer assay itself. Extensive testing of this assay system was performed prior to screening to evaluate the integrity of this approach.

In order to test the robustness and power of our medium throughput assay to identify and accurately discriminate potential dUTPase inhibitors, we utilized the Z-Factor assay performance measure as described by Zhang and colleagues (Zhang, 1999). The purpose of this assay performance measure is to assess the signal to noise ratio and determine the probability that candidate molecules with dUTPase inhibitory activity were identified by chance. As the PiPer assay is designed to quantify the concentration of pyrophosphate, we first calculated the Z-Factor based on results obtained from purified Ppi positive controls of known concentration. We then calculated the Z-Factor based on the ability to detect Ppi from a positive control generated from the hydrolysis of a known concentration of dUTP in the presence of 5 ng of recombinant dUTPase to yield dUMP and pyrophosphate. The relative fluorescence data from 63 independent screens over a twelve-month period were included in the analysis. According to the interpretation of the Z-Factor, a value of 0.92 obtained for assaying purified pyrophosphate and a Z-Factor of 0.93 for the dUTP assay indicate that both assays possess a very large dynamic range with small standard deviations. This indicates that the PiPer assay is an effective and amenable assay for high-throughput screening and importantly, that modification of the PiPer assay to include cold dUTP and recombinant dUTPase does not compromise the assays capability to accurately quantify pyrophosphate.

TABLE 3
Z-Factor calculations.

| | Positive | | Negative | | Absolute | | Z |
|---|---|---|---|---|---|---|---|
| | Control | STDev | Control | STDev^{a} | Value R | SSD* | Factor |
| PPi | 7392315.1 | 127309.7 | 739441.1 | 47039.4 | 6652874.0 | 174349.2 | 0.921 |

| | Positive | | Negative | | Absolute | | Z |
|---|---|---|---|---|---|---|---|
| | Control | STDev | Control | STDev^{a} | Value R | SSD* | Factor |
| PPi | 7392315.1 | 127309.7 | 739441.1 | 47039.4 | 6652874.0 | 174349.2 | 0.921 |

*sum of standard deviations; astandard deviation. Absolute value R refers to the dynamic range of the assay. Values represent raw fluorescence data.

Many modifications and variation of the invention as hereinbefore set forth can be made without departing from the spirit and scope thereof and therefore only such limitations should be imposed as are indicated by the appended claims.

All patent and literature references cited in the present specification are hereby incorporated by reference in their entirety.

### REFERENCES

The following references are cited herein. The entire disclosure of each reference is relied upon and incorporated by reference herein.
1. Longley D B, Harkin D P, and Johnston P G. 5-fluorouracil: mechanisms of action and clinical strategies. Nat Rev Cancer 2003;3:330-338.
2. Douillard J Y, Cunningham D, Roth A D, et al. Irinotecan combined with fluorouracil compared with fluorouracil alone as first-line treatment for metastatic colorectal cancer: a multicentre randomised trial. Lancet 2000;355:1041-1047.
3. Giacchetti S, Perpoint B, Zidani R, et al. Phase III multicenter randomized trial of oxaliplatin added to chronomodulated fluorouracil-leucovorin as first-line treatment of metastatic colorectal cancer. J Clin Oncol 2000;18:136-147.
4. Tournigand C, Andre T, Achille E, et al. FOLFIRI followed by FOLFOX6 or the reverse sequence in advanced colorectal cancer: a randomized GERCOR study. J Clin Oncol 2004;22:229-237.
5. Hurwitz H, Fehrenbacher L, Novotny W, et al. Bevacizumab plus irinotecan, fluorouracil, and leucovorin for metastatic colorectal cancer. N Engl J Med 2004;350:2335-2342.
6. Van Cutsem E, Nowacki M P, Lang I, et al. Randomized phase III study of irinotecan and 5-FU/FA with or without cetuximab in the first-line treatment of patients with metastatic colorectal cancer (mCRC): The CRYSTAL trial. J Clin Oncol 2007;2007 ASCO Annual Meeting Proceedings Part I. 25:Abstract: 4000.
7. Geyer C E, Forster J, Lindquist D, et al. Lapatinib plus capecitabine for HER2-positive advanced breast cancer. N Engl J Med 2006;355:2733-2743.
8. Thomas E S, Gomez H L, Li R K, et al. Ixabepilone plus capecitabine for metastatic breast cancer progressing after anthracycline and taxane treatment. J Clin Oncol 2007;25:5210-5217.
9. Koehler S E and Ladner R D. Small interfering RNA-mediated suppression of dUTPase sensitizes cancer cell lines to thymidylate synthase Mol Pharmacol 2004;66:620-626.
10. Sherman P A and Fyfe J A. Enzymatic assay for deoxyribonucleoside triphosphates using synthetic oligonucleotides as template primers. Anal Biochem 1989;180:222-226.
11. Horowitz R W, Zhang H, Schwartz E L, Ladner R D, and Wadler S. Measurement of deoxyuridine triphosphate and thymidine triphosphate in the extracts of thymidylate synthase-inhibited cells using a modified DNA polymerase assay. Biochem Pharmacol 1997;54:635-638.
12. Ladner R D, Lynch F J, Groshen S, et al. dUTP nucleotidohydrolase isoform expression in normal and neoplastic tissues: association with survival and response to 5-fluorouracil in colorectal cancer. Cancer Res 2000;60:3493-3503.
13. Van Cutsem E, Twelves C, Cassidy J, et al. Oral capecitabine compared with intravenous fluorouracil plus leucovorin in patients with metastatic colorectal cancer: results of a large phase III study. J Clin Oncol 2001;19:4097-4106.
14. Tyagi P and Grothey A. Commentary on a phase III trial of bevacizumab plus XELOX or FOLFOX4 for first-line treatment of metastatic colorectal cancer: the NO16966 trial. Clin Colorectal Cancer 2006;6:261-264.
15. Miwa M, Ura M, Nishida M, et al. Design of a novel oral fluoropyrimidine carbamate, capecitabine, which generates 5-fluorouracil selectively in tumours by enzymes concentrated in human liver and cancer tissue. Eur J Cancer 1998;34:1274-1281.
16. Johnston P G, Lenz H J, Leichman C G, et al. Thymidylate synthase gene and protein expression correlate and are associated with response to 5-fluorouracil in human colorectal and gastric tumors. Cancer Res 1995;55:1407-1412.
17. Lenz H J, Hayashi K, Salonga D, et al. p53 point mutations and thymidylate synthase messenger RNA levels in disseminated colorectal cancer: an analysis of response and survival. Clinical Cancer Res 1998;4:1243-1250.
18. Salonga D, Danenberg K D, Johnson M, et al. Colorectal tumors responding to 5-fluorouracil have low gene expression levels of dihydropyrimidine dehydrogenase, thymidylate synthase, and thymidine phosphorylase. Clin Cancer Res 2000;6:1322-1327.
19. Popat S, Matakidou A, and Houlston R S. Thymidylate synthase expression and prognosis in colorectal cancer: a systematic review and meta-analysis. J Clin Oncol 2004;22:529-536.
20. Horie N and Takeishi K. Functional structure of the promoter region of the human thymidylate synthase gene and nuclear factors that regulate the expression of the gene. Nucleic Acids Symp Ser 1995:77-78.
21. Pullarkat S T, Stoehlmacher J, Ghaderi V, et al. Thymidylate synthase gene polymorphism determines response and toxicity of 5-FU chemotherapy. Pharmacogenomics J 2001;1:65-70.
22. Mandola M V, Stoehlmacher J, Muller-Weeks S, et al. A novel single nucleotide polymorphism within the 5′ tandem repeat polymorphism of the thymidylate synthase gene abolishes USF-1 binding and alters transcriptional activity. Cancer Res 2003;63:2898-2904.
23. Mandola M V, Stoehlmacher J, Zhang W, et al. A 6 by polymorphism in the thymidylate synthase gene causes message instability and is associated with decreased intratumoral TS mRNA levels. Pharmacogenetics 2004;14:319-327.
24. Edler D, Glimelius B, Hallstrom M, et al. Thymidylate synthase expression in colorectal cancer: a prognostic and predictive marker of benefit from adjuvant fluorouracil-based chemotherapy. J Clin Oncol 2002;20:1721-1728.
25. Diasio R B and Harris B E. Clinical pharmacology of 5-fluorouracil. Clin Pharmacokinet 1989;16:215-237.
26. el-Hajj H H, Zhang H, and Weiss B. Lethality of a dut (deoxyuridine triphosphatase) mutation in Escherichia coli. J Bacterial 1988;170:1069-1075.
27. Curtin N J, Harris A L, and Aherne G W. Mechanism of cell death following thymidylate synthase inhibition: 2′-deoxyuridine-5′-triphosphate accumulation, DNA damage, and growth inhibition following exposure to CB3717 and dipyridamole. Cancer Res 1991;51:2346-2352.
28. Webley S D, Hardcastle A, Ladner R D, Jackman A L, and Aherne G W. Deoxyuridine triphosphatase (dUTPase) expression and sensitivity to the thymidylate synthase (TS) inhibitor ZD9331. Br J Cancer 2000;83:792-799.
29. Ingraham H A, Tseng B Y, and Goulian M. Nucleotide levels and incorporation of 5-fluorouracil and uracil into DNA of cells treated with 5-fluorodeoxyuridine. Mal Pharmacol 1982;21:211-216.
30. An Q, Robins P, Lindahl T, and Barnes D E. 5-Fluorouracil incorporated into DNA is excised by the Smug1 DNA glycosylase to reduce drug cytotoxicity. Cancer Res 2007;67:940-945.
31. Caradonna S J and Cheng Y C. The role of deoxyuridine triphosphate nucleotidohydrolase, uracil-DNA glycosylase, and DNA polymerase alpha in the metabolism of FUdR in human tumor cells. Mol Pharmacol 1980;18:513-520.
32. Tinkelenberg B A, Hansbury M J, and Ladner R D. dUTPase and uracil-DNA glycosylase are central modulators of antifolate toxicity in Saccharomyces cerevisiae. Cancer Res 2002;62:4909-4915.
33. Canman C E, Radany E H, Parsels L A, et al. Induction of resistance to fluorodeoxyuridine cytotoxicity and DNA damage in human tumor cells by expression of Escherichia coli deoxyuridinetriphosphatase. Cancer Res 1994;54:2296-2298.
34. Adlard J W, Richman S, Royston P, et al. Assessment of multiple markers for association with response rate (RR) and failure-free survival (FFS) in patients with advanced colorectal cancer (CRC) treated with chemotherapy in the MRC CR08 (FOCUS) randomized trial. J Clin Oncol, 2004 ASCO Annual Meeting Proceedings 2006;Vol 22, No 148 (July 15 Supplement) Abstract:9506.
35. Richman S, Braun M S, Adlard J W, et al. Prognostic value of thymidylate synthase (TS) expression on failure-free survival of fluorouracil-treated metastatic colorectal cancer patients. J Clin Oncol 2006;ASCO Annual Meeting Vol 24, No 18S (June 20 Supplement) Abstract: 10011.
36. Fleischmann J, Kremmer E, Muller S, et al. Expression of deoxyuridine triphosphatase (dUTPase) in colorectal tumours. Int J Cancer 1999;84:614-617.
37. Dumontet C and Sikic B I. Mechanisms of action of and resistance to antitubulin agents: microtubule dynamics, drug transport, and cell death. J Clin Oncol 1999;17:10614070.
38. Mol C D, Harris J M, McIntosh E M, and Tainer J A. Human dUTP pyrophosphatase: uracil recognition by a beta hairpin and active sites formed by three separate subunits. Structure 1996;4:1077-1092.
39. Neamati N and Barchi J J, Jr. New paradigms in drug design and discovery. Curr Top Med Chem 2002;2:211-227.
40. Jones G, Willett P, Glen R C, Leach A R, and Taylor R. Development and validation of a genetic algorithm for flexible docking. J Mol Biol 1997;267:727-748.
41. Heidelberger C, Chaudhuri N K, Danneberg P, et al. Fluorinated pyrimidines, a new class of tumour-inhibitory compounds. Nature 1957;179:663-666.
42. Aherne, G. et al. The role of uracil misincorporation in thymineless death, Anticancer Drug Development Guide: Antifolate Drugs in Cancer Therapy. A. L. Jackman. Totowa, Humana Press Inc.: 1999;409-421.
43. Barclay, B. J., B. A. Kunz, et al. “Genetic and biochemical consequences of thymidylate stress.” Canadian Journal of Biochemistry 1982;60(3): 172-84.
44. Beck, W. R., G. E. Wright, et al. (1986). “Enhancement of methotrexate cytotoxicity by uracil analogues that inhibit deoxyuridine triphosphate nucleotidohydrolase (dUTPase) activity.” Advances in Experimental Medicine & Biology 195 Pt B: 97-104.
45. Bertani, E., A. Haggmark, et al. “Enzymatic synthesis of deoxyribonucleotides: 11 Formation and intraconversion of deoxyuridine phosphates.” Journal of Biological Chemistry 1963;238: 3407-3413.
46. Canman, C. E., T. S. Lawrence, et al. “Resistance to fluorodeoxyuridine-induced DNA damage and cytotoxicity correlates with an elevation of deoxyuridine triphosphatase activity and failure to accumulate deoxyuridine triphosphate.” Cancer Research 1993;53(21): 5219-24.
47. Cohen, S. S. “On the nature of thymineless death.” Annals of the New York Academy of Sciences 1971;186: 292-301.
48. Gadsden, M. H., E. M. McIntosh, et al. “dUTP pyrophosphatase is an essential enzyme in Saccharomyces cerevisiae.” EMBO Journal 12(11): 1993;4425-31.
49. Goulian, M., B. Bleile, et al. “The effect of methotrexate on levels of dUTP in animal cells.” Journal of Biological Chemistry 1980;255(22): 10630-7.
50. Goulian, M., B. Bleile, et al. “Methotrexate-induced misincorporation of uracil into DNA.” Proceedings of the National Academy of Sciences of the United States of America 1980;77(4): 1956-60.
51. Grem, J. L. (1988). 5-Fluorouracil plus leucovorin in cancer therapy. Principals and Practice of Oncology Update Series. J. De Vita, V. T., S. Hellman and A. Rosenberg. Philadelphia, Pa., J.B. Lippincott. 2.
52. Ingraham, H. A., L. Dickey, et al. “DNA fragmentation and cytotoxicity from increased cellular deoxyuridylate.” Biochemistry 1986;25(11): 3225-30.
53. Jackman, A., et al. “The new generation of thymidylate synthase inhibitors in clinical study.” Exp Opin Invest Drugs 1996; 5: 719-736.
54. Jackson, R. C. and G. B. Grindey (1984), The biochemical basis for methotrexate cytotoxicity. In: Folate antagonists as therapeutic agents. 1, Biochemistry, molecular actions, and synthetic design. New York, Academic.
55. Kunz, B. A., S. E. Kohalmi, et al. “Deoxyribonucleoside triphosphate levels: a crtical factor in the maintenance of genetic stability.” Mutation Research 1994;318: 1-64.
56. Ladner, R. D. “The role of dUTPase and uracil-DNA repair in cancer chemotherapy.” Curr Protein Pept Sci 2001;2(4): 361-70.
57. Lindahl, T. “DNA repair enzymes.” Annual Review of Biochemistry 1982;51: 61-87.
58. Lindahl, T. “Instability and decay of the primary structure of DNA [see comments].” Nature 1993;362(6422): 709-15.
59. Mader, R. M., M. Muller, et al. “Resistance to 5-fluorouracil.” General Pharmacology 1998;31(5): 661-6.
60. McIntosh, E. M. and R. H. Haynes “dUTP pyrophosphatase as a potential target for chemotherapeutic drug development.” Acta Biochimica Polonica 1997;44(2): 159-71.
61. Mol, C. D., J. M. Harris, et al. “Human dUTP pyrophosphatase: uracil recognition by a beta hairpin and active sites formed by three separate subunits.” Structure 1996;4(9): 1077-92.
62. Parsels, L. A., J. D. Parsels, et al. “Mechanism and pharmacological specificity of dUTPase-mediated protection from DNA damage and cytotoxicity in human tumor cells.” Cancer Chemotherapy & Pharmacology 1998;42(5): 357-62.
63. Pu, W. T. and K. Struhl “Uracil interference, a rapid and general method for defining protein-DNA interactions involving the 5-methyl group of thymines: the GCN4-DNA complex.” Nucleic Acids Research 1992;20(4): 771-5.
64. Richards, R. G., O. E. Brown, et al. “Drug concentration-dependent DNA lesions are induced by the lipid-soluble antifolate, piritrexim (BW301U).” Molecular Pharmacology 1986;30(6): 651-8.
65. Sedwick, W. D., M. Kutler, et al. “Antifolate-induced misincorporation of deoxyuridine monophosphate into DNA: inhibition of high molecular weight DNA synthesis in human lymphoblastoid cells.” Proceedings of the National Academy of Sciences of the United States of America 1981;78(2): 917-21.
66. Webley, S. D., S. J. Welsh, et al. “The ability to accumulate deoxyuridine triphosphate and cellular response to thymidylate synthase (TS) inhibition.” Br J Cancer 2001;85(3): 446-52.
67. Zhang, J. H., T. D. Chung, and K. R. Oldenburg, A Simple Statistical Parameter for Use in Evaluation and Validation of High Throughput Screening Assays. J Biomol Screen, 1999. 4(2): p. 67-73.

What is claimed is:

1. A composition comprising a deoxyuridine triphosphosphate nucleotidohydrolase (dUTPase) inhibitor that decreases the expression of dUTPase and increases the ability of the fluoropyrimidine class of anticancer agents to break DNA strands and thereby increases the cytotoxicity of cancer cells.

2. The composition according to claim 1, wherein said fluoropyrimidine class of anticancer agent is 5-flurouracil (5-FU).

3. The composition according to claim 1, wherein said dUTPase inhibitor is DU7, DUG, BB37, or DU44.

4. A method of increasing the cytotoxicity of a cancer cell by contacting said cancer cell with a composition comprising a dUTPase inhibitor, wherein said cancer cell exhibits dUTPase overexpression.

5. The method according to claim 4 wherein said cancer cell is breast or colon.

6. The method according to claim 4, wherein said dUTPase inhibitor is DU7, DU6, BB37, or DU44.

7. A composition comprising a dUTPase inhibitor that directly inhibits dUTPase and promotes dUTP accumulation and uracil-misincorporation thereby synergistically improving the clinical efficacy of thymidylate synthase (TS)-directed chemotherapy.

8. The composition according to claim 7, wherein said TS-directed chemotherapy comprises 5-flurouracil (5-FU).

9. The composition according to claim 7, wherein said dUTPase inhibitor is DU7 DU6, BB37, or DU44.

10. A method of synergistically improving the clinical efficacy of thymidylate TS-directed chemotherapy comprising contacting a cancer cell with a composition comprising a dUTPase inhibitor, wherein said cancer cell exhibits dUTPase overexpression.

11. The method according to claim 10, wherein said dUTPase inhibitor is DU7, DU6, BB37, or DU44.

12. A composition comprising a dUTPase inhibitor that directly inhibits dUTPase and promotes dUTP accumulation thereby decreases TS-directed drug resistance.

13. The composition according to claim 12, wherein said TS-directed drug resistance is to the fluoropyrimidine class of anticancer agents.

14. The composition according to claim 13, wherein said fluoropyrimidine anticancer agent is 5-flurouracil (5-FU).

15. The composition according to claim 12, wherein said dUTPase inhibitor is DU7, DU6, BB37, or DU44.

16. A method of decreasing TS-directed drug resistance comprising contacting a cancer cell with a composition comprising a dUTPase inhibitor, wherein said cancer cell exhibits dUTPase overexpression.

17. The method according to claim 16, wherein said TS-directed drug resistance is to the fluoropyrimidine class of anticancer agents.

18. The method according to claim 12, wherein said fluoropyrimidine anticancer agent is 5-flurouracil (5-FU).

19. The method according to claim 16, wherein said dUTPase inhibitor is DU7, DU6, BB37, or DU44.

20. A composition comprising a dUTPase inhibitor that synergistically improves the efficacy of 5-fluorouracil (5-FU)-based chemotherapies in a wide variety of cancers, wherein said wide variety of cancers exhibits an overexpression of dUTPase.

21. The composition according to claim 20, wherein said dUTPase inhibitor is DU7, DU6, BB37, or DU44.

22. A method of synergistically improving the efficacy of 5-fluorouracil (5-FU)-based chemotherapies in a wide variety of cancers comprising contacting said variety of cancer cells with a composition comprising a dUTPase inhibitor and wherein said variety of cancer cell exhibits dUTPase overexpression.

23. The method according to claim 23, wherein said dUTPase inhibitor is DU7, DU6, BB37, or DU44.

24. A method of screening for dUTPase inhibitors comprising:
generating a pharmacophore model of the dUTPase enzyme bound to a dUDP substrate;
perform docking studies with the dUTPase pharmacophore model against potential dUTPase antagonists;
predict docking scores of potential dUTPase antagonists; and
perform enzyme assay with candidate molecules displaying efficient contact with the dUTPase active site.
